(19) United States
(12) Patent Application Publication
(10) Pub. No.: US 2026/0112037 A1
(43) Pub. Date: Apr. 23, 2026

(54) SYSTEMS AND METHODS FOR UTILIZING INTERFEROMETRIC ULTRA-HIGH RESOLUTION 3D IMAGING

(71) Applicant: Purdue Research Foundation, West Lafayette, IN (US)

(72) Inventors: Fang Huang, West Lafayette, IN (US); Hao-Cheng Gao, West Lafayette, IN (US); Fan Xu, West Lafayette, IN (US)

(21) Appl. No.: 19/349,551

(22) Filed: Oct. 3, 2025

Related U.S. Application Data
(60) Provisional application No. 63/703,144, filed on Oct. 3, 2024.

(51) Int. Cl. G06T 7/00; A61B 90/20; G06T 7/10; G06T 7/73

(52) U.S. Cl. CPC G06T 7/0016; G06T 7/10; G06T 7/73; A61B 90/20; G06T 2200/04; G06T 2207/10056; G06T 2207/30012; G06T 2207/30016

(57) ABSTRACT

The invention generally relates to systems and methods for utilizing interferometric ultra-high resolution 3D imaging. In certain aspects, the invention provides a system for achieving molecular resolution through tissues, the system comprising: an interferometric apparatus; and a control unit comprising one or more processors configured to: receive interferometric tissue data from the interferometric apparatus, wherein the interferonic tissue data comprises one or more tissue-induced abberations; apply a 4Pi in-situ point spread function (PSF) retrieval algorithm to the interferonic tissue data, thereby enabling direct retrieval of an aberrated, time-dependent in-situ model of single-molecule emission pattern from the interferonic tissue data, wherein the in-situ model reflects distortions, blurring, and/or partial coherence encountered during detection; and minimize disparities between the interferonic tissue data and the in-situ model to thereby provide accurate molecular localizations in the presence of tissue-induced aberrations.

### RELATED APPLICATION

The present application claims the benefit of and priority to U.S. provisional patent application Ser. No. 63/703,144, filed Oct. 3, 2024, the content of which is incorporated by reference herein in its entirety.

### GOVERNMENT SUPPORT

This invention was made with government support under R35GM119785 awarded by the National Institutes of Health. The government has certain rights in the invention.

### FIELD OF THE INVENTION

The invention generally relates to systems and methods for utilizing interferometric ultra-high resolution 3D imaging.

### BACKGROUND

The brain, a network comprising billions of interconnected neurons, forms the basis for cognitive development. A neural circuit, the basic functional unit of the brain, consists of neurons interconnected by synapses where information flows from one neuron to another. The synapses facilitate the transmission of neurotransmitters from presynaptic terminals to postsynaptic spines. Understanding the nano-anatomy of dendritic spines is crucial for comprehending brain development and higher functions. These spines, which house the postsynaptic components of most excitatory synapses, change in size throughout life, reflecting synaptic strength and plasticity during development and aging. Both the morphology of spine heads and the spine necks play critical roles in synapse transmission by regulating chemical and electrical compartmentalization. However, studying these protein complexes, organized at a nanometer scale of 15-60 nm inside thick tissues, poses challenges for conventional microscopy systems.

Due to the wave nature of light, the resolution of conventional fluorescence microscopy methods is limited to sub-micrometer level, inadequate to visualize synaptic ultrastructure and molecular distributions. Recent advancements in super-resolution microscopy techniques, such as stimulated emission depletion (STED) microscopy, single-molecule switching nanoscopy (SMSN), and structured illumination microscopy (SIM), have enhanced resolution by nearly tenfold. These techniques have empowered researchers to probe protein organization within presynaptic boutons and postsynaptic densities, primarily in isolated neuronal cultures.

Images of isolated neuron cultures have shed light on the spatial arrangements of proteins, synaptic interactions, and neuronal communication, deepening our understanding of neural circuitry. However, the extraction of individual neurons from their native tissue context compromises the structural and functional integrity of neural connections, hindering the investigation of synaptic regulation during information processing and memory formation. To uncover the molecular mechanisms of the circuit function in the native or disease contexts, direct examination inside brain tissue is imperative. Yet, resolving the molecular components within brain tissues is challenging due to the complex optical properties of intra-and-extra cellular constituents. The accurate retrieval of aberration in situ is critically important on the resolvability of nano-architecture within brain tissue which affects all super-resolution modalities. Moreover, conventional 3D super-resolution microscopy techniques exhibit pronounced disparities between axial and lateral resolutions, especially through deep tissues, and are limited to a 3D resolution of ˜85-120 nm in brain sections of 50 μm or thicker.

### SUMMARY

The invention recognizes that to unravel the molecular intricacies of the brain, an optical imaging platform must attain and sustain an isotropic 3D resolution of sub-15 nm in unperturbed tissue specimens while minimizing distortions and artifacts. To that end, the invention generally relates to a 4Pi single-molecule nanoscopy for brain with in-situ point spread function retrieval through opaque tissue (4Pi-BRAINSPOT). This approach allows an in-situ localization precision of 6 nm laterally and 2.5 nm axially through 50-μm brain slices by synergizing the isotropic 3D resolution capabilities of 4Pi single-molecule switching nanoscopy (4Pi-SMSN) with a novel in-situ coherent PSF retrieval approach, SMSN-compatible tissue clearing, and highly inclined light-sheet illumination. It is demonstrated herein that 4Pi-BRAINSPOT enables 3D nanoarchitecture visualization through thick brain tissues, revealing the molecular distribution and ultrastructure of dendritic spines while preserving circuit integrity unattainable in cultured neurons. The invention additionally provides a novel analysis pipeline for quantifying spine morphology utilizing the highly accurate molecular locations obtained by 4Pi-BRAINSPOT. It is believed that 4Pi-BRAINSPOT will enable molecular-level investigation of synaptic regulation within neurobiological processes and thus enhancing our understanding of the mechanisms underlying various neurodevelopmental disorders.

In certain aspects, the invention provides systems for achieving molecular resolution through tissues that include an interferometric apparatus; and a control unit. The control unit includes one or more processors configured to: receive interferometric tissue data from the interferometric apparatus, wherein the interferonic tissue data comprises one or more tissue-induced abberations; apply a 4Pi in-situ point spread function (PSF) retrieval algorithm to the interferonic tissue data, thereby enabling direct retrieval of an aberrated, time-dependent in-situ model of single-molecule emission pattern from the interferonic tissue data, wherein the in-situ model reflects distortions, blurring, and/or partial coherence encountered during detection; and minimize disparities between the interferonic tissue data and the in-situ model to thereby provide accurate molecular localizations in the presence of tissue-induced aberrations.

In other aspects, the invention provides methods for achieving molecular resolution through tissues that involve receiving to a processor interferometric tissue data from an interferometric apparatus, wherein the interferonic tissue data comprises one or more tissue-induced abberations; applying via the processor a 4Pi in-situ point spread function (PSF) retrieval algorithm to the interferonic tissue data, thereby enabling direct retrieval of an aberrated, time-dependent in-situ model of single-molecule emission pattern from the interferonic tissue data, wherein the in-situ model reflects distortions, blurring, and/or partial coherence encountered during detection; and minimizing via the processor disparities between the interferonic tissue data and the in-situ model to thereby provide accurate molecular localizations in the presence of tissue-induced aberrations.

In certain embodiments of the systems and methods herein, the 4Pi in-situ PSF retrieval algorithm comprises segmenting multichannel 4Pi emission patterns to create a library of detected emission patterns. In certain embodiments of the systems and methods herein, the segments, from stochastically blinking events at varying axial positions, serve as random samples of underlying in-situ interferometric (4Pi) PSF that are desired to be retrieved. In certain embodiments of the systems and methods herein, wherein to estimate axial positions of patterns in the library, reference PSFs are generated from coherent pupil functions, depicting interferometric amplitude and phase of wavefield superposition from the pupils of two opposing objectives.

In certain embodiments of the systems and methods herein, wherein detected emission patterns are then assigned to specific axial groups according to their similarity to the reference PSFs. In certain embodiments of the systems and methods herein, wherein categorized patterns are then aligned and averaged within each group to generate experimental PSF observations. In certain embodiments of the systems and methods herein, wherein to retrieve individual pupil functions, a coherent 4Pi phase retrieval algorithm allows retrieval of the individual pupil functions for each objective from their coherent superpositions and updates the reference 4Pi-PSFs for the subsequent iteration.

In certain embodiments of the systems and methods herein, wherein through sequential iterations, the model is refined to capture detailed coherent aberrations from both instrumental imperfections and biological specimens, representing in-situ interferometric emission patterns for accurate and precise single-molecule localization inside tissue specimens. In certain embodiments of the systems and methods herein, the interferonic tissue data comprises data from brain tissue slices that are at least 50-μm thick. In certain embodiments of the systems and methods herein, the system and/or methods provide an isotropic 3D resolution of sub-15 nm in the brain tissue slices while minimizing distortions and artifacts.

### BRIEF DESCRIPTION OF THE DRAWINGS

FIG. 1 panels A-H provide an overview of 4Pi-BRAINSPOT concepts and demonstrations. (Panel A) Imaging workflow of 4Pi-BRAINSPOT. To image opaque brain tissues, we first excised, fixed, and sectioned the tissues into 50-μm coronal sections. These sections underwent immunofluorescence-labeling targeting the protein of interest, followed by tissue clearing, and were mounted with thiol-based imaging buffer. Imaging is conducted with 4Pi-SMSN, utilizing a 54°-inclined light-sheet illumination. The obtained single-molecule datasets were processed with the algorithm detailed in (Panel B), which extracted dynamic in-situ retrieval of the 4Pi PSF model and facilitated the 3D localization of single molecules for detailed visualization and quantification. (Panel B) Data processing workflow of in-situ PSF modeling in 4Pi-BRAINSPOT. The algorithm segmented individual 4Pi emission patterns from single-molecule datasets to create a library. These segments were assigned to specific axial groups according to their similarity to the reference PSFs generated from initial coherent pupil functions. The categorized patterns were then aligned and averaged within each group and underwent the coherent 4Pi phase retrieval algorithm, updating the individual pupil functions and reference PSFs. Through sequential iterations, the model was refined to represent in-situ interferometric emission patterns. The model was fed into the dynamic model update in (Panel C), capturing time-varying response functions. (Panel C) Data processing workflow of dynamic in-situ model update in 4Pi-BRAINSPOT. We generated a series of reference PSFs for each time window based on coherent pupil functions from the previous window, with adjustments of 3D objective misalignments (AX, AY, and AZ) and cavity phase shifts (90). The algorithm iteratively refined the time-dependent parameters to capture the alterations in single-molecule patterns according to the similarity between the PSF model and segmented emission patterns in the current time window. (Panels D and E) Interferometric pattern comparison between fluorescent bead measurements (top row) and 4Pi-BRAINSPOT PSF models (bottom row) in channel p1 with two different aberrations at axial positions ranging from ±1 μm. Retrieved coherent pupil functions are shown on the right. (Panel F) Tissue-clearing results between mouse brain slices immersed in PBS and tissue-cleared mouse brain slices. Box size: 2.5 mm. (Panel G) Gaussian-weighted moment operators of 4Pi interference of fluorescent bead measurement with PBS-immersed (left) and tissue-cleared (right) mouse brain slices. (Panel H) Comparison of coherence contrast, signal-to-background ratio (SBR), and localization precisions between PBS-immersed (black) and tissue-cleared (orange) mouse brain slices. Bar height is the mean and error bar is the standard deviation. Statistical significance: t-test.

FIG. 2 panels A-I show performance quantification of 4Pi-BRAINSPOT. (Panel A) Simulated 4Pi single-molecule emission patterns in channel p1 with known aberrations at random axial positions ranging from +800 nm. (Panel B) Comparison of interferometric wavefield superpositions at the axial position of −800 nm in (Panel A) retrieved using 4Pi-BRAINSPOT (top) and the ground truth (bottom). (Panel C) Horizontal and vertical cross-sections of PSFs of the 4Pi-BRAINSPOT (top row), in-vitro method (middle row), and ground truth (bottom row). (Panel D) NCC similarity between ground truth PSFs and modeled PSFs using 4Pi-BRAINSPOT (orange) and in-vitro method (black). Boxes are upper and lower quartiles, central lines are median, and error bars are furthest data points excluding outliers (n=30 trials for each). Statistical significance: t-test. (Panel E) Scatter plot of ground-truth lateral objective misalignment versus retrieved objective misalignment in simulations using dynamic in-situ model update with coherent pupil functions retrieved using 4Pi-BRAINSPOT (orange) and in-vitro method (black) (n=60 trials for each). (Panel F) Scatter plot of ground-truth axial objective misalignment versus retrieved objective misalignment in simulations using dynamic in-situ model update with coherent pupil functions retrieved using 4Pi-BRAINSPOT (orange) and in-vitro method (black) (n=60 trials for each). (Panel G) Scatter plot of ground-truth phase cavity versus retrieved objective misalignment in simulations using dynamic in-situ model update with coherent pupil functions retrieved using 4Pi-BRAINSPOT (orange) and in-vitro method (black) (n=60 trials for each). (Panel H) Bias of cavity phase estimation with deviated initial guess of coherent pupils of 0, π/4, π/2, and 3π/4 (n=30 trials for each). (Panel I) Goodness of fitting between the obtained emission patterns and modeled PSFs of AF647-labeled TOM20 in U2OS cells using 4Pi-BRAINSPOT (orange) and in-vitro method (black). Random aberrations were introduced via deformable mirrors for each trial. Box is the upper and lower quartiles, central line is the median, and error bar is the furthest data point excluding outliers. Statistical significance: t-test.

FIG. 3 panels A-U show performance of 4Pi-BRAINSPOT in mammalian cells. (Panel A) Reconstruction of AF647-labeled a-tubulin in COS-7 cells with 25-μm cavity, color-coded according to single-molecule axial positions. (Panels B-M) Vertical cross-sections of selected positions (white dashed lines) in (Panel A). (Panels N and O) Comparisons of vertical cross-sections between 4Pi-BRAINSPOT, in-vitro voxel-based method using beads on top coverslips (Voxel (top)), in-vitro voxel-based method using beads on bottom coverslips (Voxel (bot.)), in-vitro pupil-based method using beads on top coverslips (Pupil (top)), and in-vitro pupil-based method using beads on bottom coverslips (Pupil (bot.)). (Panel P) Normalized intensity profile along white dashed lines in (N). Yellow bar is the localization histogram with 6-nm bins and red curve is the fitted Gaussian function to the histogram. Features isolated or below the exclusion threshold (gray dashed line) are excluded from analysis. Black dashed line marks the estimated feature center, solid arrowhead indicates excluded isolated features, and dashed arrowhead indicates feature discontinuity. (Panel Q) Reconstruction of AF647-labeled TOM20 in COS-7 cells with 25-μm cavity with horizontal and vertical cross-sections of selected positions (yellow dashed lines). (Panels R-T) Vertical cross-sections (left) of targeting mitochondria (white dashed lines) in (Panel Q). Normalized intensity profile (right) along white dashed lines in (Panels R-T). Yellow bar is the localization histogram with 6-nm bins, red curve is fitted Gaussian functions to the histogram, gray dashed line is the exclusion threshold, and black solid line marks the estimated feature FWHM. (Panel U) Horizontal (left) and vertical (middle) cross-sections and normalized intensity profiles (right) of superposed single-molecule cluster. Red dot is single-molecule localization (n=1845 localizations), grayscale distribution is rendered cluster using 2D Gaussian blur with estimated FWHM, orange curve is fitted Gaussian function, and blue line marks estimated FWHM.

FIG. 4 panels A-M show demonstration of endoplasmic reticulum (ER) tubules in mammalian cells using 4Pi-BRAINSPOT. (Panel A) Reconstruction of AF647-labeled Rtn-4 in COS-7 cells, color-coded according to single-molecule axial positions. (Panel B) Illustration of ER tubules with elliptical lumens (top), where Rtn-4 (magenta) concentrates on the high-curvature edges only, and round lumens (bottom), where Rtn-4 distributed across the surface. (Panel C and D) Vertical cross-sections of selected positions (white dashed lines) in (Panel A) showing ring structures. White dashed circle marks the estimated ring structure and white solid line indicates its radius. (Panels E-G) Vertical cross-sections of selected positions (white dashed lines) in (A) showing parallel lines. White dashed circle marks estimated double-line structure and white solid line indicates interval distance. (Panel H) Radius analysis in (Panel D). Blue bar is the distance histogram between localizations and the structural center with 4-nm bins, red curve is fitted Gaussian functions to the histogram, and black dashed line marks the estimated radius. (Panel I) Interval distance analysis in (Panel F). Yellow bar is the localization histogram with 6-nm bins, red curve is fitted Gaussian functions to the histogram, gray dashed line is the exclusion threshold, black dashed line marks the estimated feature center, and black solid line indicates the estimated interval distance. (Panel J) Comparison of resolution analysis using different methods, including lateral and axial estimated resolutions derived by 2.355*✓CRLB (EstXY of 9.7±3.1 nm and EstZ of 4.0±1.3 nm, corresponding to xxx, n=2955 localizations), FSC resolution (FSC of 16.6±1.3 nm, n=81 subregions), imaging decorrelation resolution (Decor. of 13.9±1.0 nm, n=81 subregions), lateral and axial DFC resolutions (DFCXY of 16.1 nm and DFCZ of 13.8 nm), and measured lateral and axial FWHMs of superposed single-molecule cluster (FWHMXY of 8.5 nm and FWHMZ of 4.7 nm). Box is the upper and lower quartiles, central line is the median, error bar is the furthest data point excluding outliers, gray dashed line is the estimated isotropic 3D estimated resolution, and the distribution is shown on the right side. (Panel K) Workflow of circumference analysis. We first sliced tubular structures along the longitudinal direction and flattened them as 2D surfaces. Localizations are then color-coded according to angular positions ranging from ±π. (Panel L) Circumference analysis of targeting ER tubule in (Panel A). (Panel M) Vertical cross-sections of selected positions (corresponding colors) in (Panel L), showing varied distributions of Rtn-4 from double-line (red and green) to ring (magenta and yellow) structures.

FIG. 5 panels A-H show demonstration of 4Pi-BRAINSPOT in transgenic mouse brain slices. (Panel A) Reconstruction of AF647-labeled ChR2 in 50-μm thick transgenic mouse brain slices using 4Pi-BRAINSPOT, color-coded according to single-molecule axial positions. Inset shows enlarged dendritic spines in white solid box. (Panels B and C) Horizontal (Panel B) and vertical (Panel C) cross-sections (left) and normalized intensity profiles (right) of superposed single-molecule cluster. Red dot is single-molecule localization (n=4272 localizations), grayscale distribution is rendered cluster using 2D Gaussian blur with estimated FWHM, orange curve is fitted Gaussian function, and blue line marks estimated FWHM. (Panel D) Heatmap of image decorrelation resolutions by segmenting 2D projection of (A) into 7×7 subregions. (Panels E and F) Vertical cross-sections of targeting dendritic spines (white dashed lines) in (Panel A) using 4Pi-BRAINSPOT (left), in-vitro voxel-based method (middle), and in-vitro pupil-based method (right). (Panel G) Normalized intensity profile along white dashed lines in (Panel E). Yellow bar is the localization histogram with 6-nm bins, red curve is fitted Gaussian functions to the histogram, gray dashed line is the exclusion threshold, black solid line marks the estimated feature FWHM, solid arrowhead indicates excluded isolated features, and dashed arrowhead indicates feature discontinuity. (Panel H) Comparison of resolution analysis using different methods, including lateral and axial estimated resolutions (EstXY of 13.3+2.6 nm and EstZ of 5.8+1.5 nm, n=4272 localizations), FSC resolution (FSC of 22.2+3.6 nm, n=81 subregions), imaging decorrelation resolution (Decor. of 15.7+0.4 nm, n=81 subregions), lateral and axial DFC resolutions (DFCXY of 24.9 nm and DFCZ of 17.7 nm), and measured lateral and axial FWHMs of superposed single-molecule cluster (FWHMXY of 11.1 nm and FWHMZ of 5.8 nm). Box is the upper and lower quartiles, central line is the median, error bar is the furthest data point excluding outliers, gray dashed line is the estimated isotropic 3D estimated resolution, and the distribution is shown on the right side.

FIG. 6 panels A-I show demonstration of spine morphological analysis. (Panel A) Reconstruction of AF647-labeled ChR2 in 50-μm thick transgenic mouse brain slices using 4Pi-BRAINSPOT, color-coded according to single-molecule axial positions. (Panels B-G) Workflow of analysis pipeline of 3D structural morphology. (Panel B) Identification of targeting spine (white boxes) in (Panel A). Yellow curve is the trace of algorithm from manual identification. (Panel C) Straighten structure of the dendritic spine in (Panel B) by segmenting localizations into 150-nm segments with 50-nm overlapping. (Panel D) Vertical cross-sections of selected positions (white dashed lines with colored arrowheads) in (Panel C) showing spine neck (green) and spine head (red). (Panel E) 3D model of the spine in (Panel B). (Panel F) Localization distribution at selected positions (white dashed lines with colored arrowheads) in (Panel C). Black dot is localization, green area marks the alpha-shaped boundary, and red cross marks the estimated structural center. (Panel G) Volume quantification of dendritic spine in (Panel C). Blue dot is the measured area and yellow curve is the fitted curve. Colored arrowhead indicates the area corresponding in (Panel C). (Panels H and I) Demonstrations of spine analysis of targeting spines (white boxes) in (Panel A) with volume quantification (right). Inset is a 3D model of the spine, green and red box are vertical cross-sections of selected positions of the spine neck and head pointed by colored arrowheads. (Panel I) Distribution of diameters of spine necks and spine heads (n=33 spines, N=16 tissues), color-coded according to spine length.

FIG. 7 panels A-G show interferometric pattern comparison between fluorescent bead measurements (top row) and 4Pi-BRAINSPOT PSF models (bottom row) in channel p1 with different aberrations at axial positions ranging from #1 μm. Retrieved coherent pupil functions are shown on the right. FIG. 7 panel H shows a heatmap of NCC similarity between measured and modeled 4Pi-PSFs in (Panels A-G). FIG. 7 panel I shows stepwise localizations of bead measurements with 100 nm increments and 50 measurements per step over an axial range from #1 μm using 4Pi-BRAINSPOT. Orange dot is localization result of 4Pi-BRAINSPOT, and gray dot is assigned stage position. Inset shows enlarged result in cyan box. FIG. 7 panel J shows Estimation of bias (top row) by calculating mean distances of localization results from assigned stage positions and precision (bottom row) by calculating standard deviations of localization results in (I). Orange curve is localization result, and gray dashed line is the stage accuracy.

FIG. 8 panels A-B show reconstruction and cross-sections of selected positions (yellow dashed line) of AF647-labeled ChR2 in PBS-immersed (Panel A) and tissue-cleared (Panel B) 50-μm thick mouse brain slices using 4Pi-BRAINSPOT, color-coded according to the single-molecule Z positions. Signal-to-background ratio (SBR) of tissue-cleared specimens was 106±15 (n=12 datasets) and that of untreated specimens was 80±6 (n=6 datasets), respectively. FIG. 8 panels C—H show enlarged views of selected regions (white dashed boxes) in (A&B). The lateral and axial localization of tissue-cleared specimens are 6.4±0.4 and 2.9±0.4 (n=12 datasets), while those of untreated specimens are 8.3±0.1 and 5.4±0.2 (n=6 datasets).

FIG. 9 shows coherent 4Pi phase retrieval began with initial coherent pupil functions with predetermined parameters. Superposed wavefields were generated by adding variable defocuses and phase delays across channels at axial positions ranging from ±800 nm. Amplitudes of 4Pi-PSF were computed by Fourier transform of superposed wavefield. Next, calculated magnitudes were replaced with the square root of obtained values, while maintaining original phases. Inverse Fourier transform was used to update superposed wavefields after which coherent pupil functions were updated by Moore-Penrose pseudo-inverse process. Results after 60 iterations were regarded as coherent pupil functions of obtained interferometric patterns.

FIG. 10A panel A shows illustration of dynamic model update in each time window. 4Pi-BRAINSPOT estimated and accommodated time-dependent interferometric aberrations according to objective misalignments (AX, AY, and AZ) and cavity phase shift (φ0). FIG. 10A panel B shows data processing workflow of dynamic in-situ model update in 4Pi-BRAINSPOT. The algorithm segmented individual 4Pi emission patterns from single-molecule datasets to create a library. We generated a series of reference PSFs for each time window based on coherent pupil functions from the previous window, with adjustments of 3D objective misalignments (AX, AY, and AZ) and cavity phase shifts (φ0). For each reference, NCC similarity score between obtained patterns and reference 4Pi-PSFs are evaluated to determine the time-dependent parameters. For objective misalignments, the corresponding coefficients are determined by fitting the similarity scores with a Gaussian function, identifying the peak position. For cavity phase variation, the corresponding coefficients are determined by periodic-padding Gaussian fitting the similarity scores and identifying the peak position. Refined results are regarded as retrieved coherent pupil functions for a given time window. FIG. 10B panel C shows data processing workflow of 3D channel-specific single-molecule localization. The process began with mapping coordinates in reference channel p1 into the other channels using calculated affine matrices. Channel-specific 4Pi-PSF models representing respective statistical properties of SCOMS are generated based on dynamic in-situ 4Pi-PSF model and used in further 4Pi localization. FIG. 10B panel D shows comparison of localization bias and precision between 4Pi-BRAINSPOT using channel-specific 4Pi-PSF models (orange) and translating acquired patterns (blue) instead. Insets are the simulated variance distribution.

FIG. 11 panel A shows reconstruction of AF647-labeled TOM20 in COS-7 cells in FIG. 3Q, color-coded according to the single-molecule Z positions. FIG. 11 panel B shows illustration of single-molecule cluster identification criteria. Magenta ellipse is region within three times of localization precision, yellow ellipse is region between three and five times of localization precision. Clusters were considered to originate from single molecules if more than three localizations fall in magenta region and none fall in yellow region. Red dot is simulated 7 localizations originated from single molecule and grayscale distribution is rendered cluster using 2D Gaussian blur. FIG. 11 panel C shows cross-sectional views cluster superposition (n=3244 localizations). Red dots are single-molecule localizations, grayscale distributions are rendered images of localization cluster, and blue lines are FWHMs of localization distributions. Horizontal (top) and vertical (bottom) cross-sections of superposed single-molecule cluster. Red dot is single-molecule localization (n=3244 localizations) and grayscale distribution is rendered cluster using 2D Gaussian blur with estimated FWHM. FIG. 11 panel D shows horizontal (top) and vertical (bottom) normalized intensity profiles of superposed single-molecule cluster. Red dot is single-molecule localization (n=3244 localizations), orange curve is fitted Gaussian function, and blue line marks estimated FWHM. FIG. 11 panel E shows lateral and axial FWHMs of all single-molecule clusters (10.3±3.0 and 5.3±1.9 nm, n=387 clusters). Gray error bar indicates mean and standard deviation of lateral and axial FWHMs. FIG. 11 panel F shows histograms of localization counts per cluster (top) and photon counts per cluster (bottom). FIG. 11 panels G-J show cross-sections and normalized intensity profiles for four selected clusters in (E) with >7 localizations each. Red dot is single-molecule localization (n=1845 localizations), grayscale distribution is rendered cluster using 2D Gaussian blur with estimated FWHM, orange curve is fitted Gaussian function, and blue line marks estimated FWHM.

FIG. 12 panel A shows reconstruction and cross-sections of selected positions (white dashed lines) of AF647-labeled a-tubulin in COS-7 cells with a 25-μm cavity using 4Pi-BRAINSPOT, color-coded according to the single-molecule Z positions. FIG. 12 panel B shows heatmap of image decorrelation resolutions by segmenting 2D projection of (Panel A) into 6×6 subregions. FIG. 12 panel C shows correlation analysis of Fourier shell correlation (FSC, orange) and directional Fourier correlation laterally and axially (DFCXY, purple; DFCZ, navy) at different spatial frequencies. Marker is correlation coefficient at selected spatial frequency, solid line is cubic-spine-fitted curve, and gray dashed line is 1/7 criteria to determine resolutions. FIG. 12 panel D shows comparison of resolution analysis using different methods (n=10 datasets, including 4 datasets of AF647-labeled a-tubulin, 3 datasets of TOM20, and 3 datasets of Rtn4 in COS-7 cells), including lateral and axial estimated resolutions (EstXY of 11.0=1.4 nm and EstZ of 4.8±0.4 nm, red), FSC resolution (FSC of 19.8±2.0 nm, orange), imaging decorrelation resolution (Decor. of 14.2±1.3 nm, green), lateral and axial DFC resolutions (DFCXY of 20.8±3.1 nm and DFCZ of 16.7±2.5 nm, purple), and measured lateral and axial FWHMs of superposed single-molecule cluster (FWHMXY of 9.9±1.2 nm and FWHMZ of 5.1±0.3 nm, blue). Central line is mean, error bar is standard deviations, and gray dashed line is estimated isotropic 3D estimated resolution.

FIG. 13 panel A shows reconstruction of AF647-labeled ChR2 in 50-μm thick mouse brain slices in FIG. 6 panel A, color-coded according to the single-molecule Z positions. FIG. 13 panels B-C show vertical cross-sections (top) of targeting dendritic spines (white dashed lines) in (Panel A). Normalized intensity profile (bottom) along white dashed lines. Yellow bar is localization histogram with 6-nm bins, red curve is fitted Gaussian functions to the histogram, gray dashed line is exclusion threshold, black solid line marks estimated feature FWHM, solid arrowhead indicates excluded isolated features, and dashed arrowhead indicates feature discontinuity. FIG. 13 panel D shows heatmap of image decorrelation resolutions by segmenting 2D projection of (A) into 7×7 subregions. FIG. 13 panel E shows comparison of resolution analysis using different methods, including lateral and axial estimated resolutions (EstXY of 14.4±3.7 nm and EstZ of 6.4±1.6 nm, n=3244 localizations, red), FSC resolution (FSC of 28.3±3.6 nm, n=81 subregions, orange), imaging decorrelation resolution (Decor. of 17.4±0.4 nm, n=81 subregions, green), lateral and axial DFC resolutions (DFCXY of 28.7 nm and DFCZ of 23.5 nm, purple), and measured lateral and axial FWHMs of superposed single-molecule cluster (FWHMXY of 12.8 nm and FWHMZ of 6.4 nm, cyan). Box is upper and lower quartiles, central line is median, error bar is furthest data point excluding outliers, gray dashed line is estimated isotropic 3D estimated resolution, and distribution is shown on the right side. FIG. 13 panels F-G show horizontal (Panel F) and vertical (Panel G) cross-sections (left) and normalized intensity profiles (right) of superposed single-molecule cluster. Red dot is single-molecule localization (n=3244 localizations), grayscale distribution is rendered cluster using 2D Gaussian blur with estimated FWHM, orange curve is fitted Gaussian function, and blue line marks estimated FWHM.

FIG. 14A panel A shows 4Pi-PSFs of channel p1 at z positions ranging from +1 μm with vertical astigmatism variations of ±1λ/2π (case 1, top row) and ∓1λ/2π (case 2, middle row) in the upper and lower pupil functions, respectively. Yellow and cyan boxes highlight identical PSFs with opposite Z positions in cases 1 and 2. Overlapping 4Pi-PSFs with axial symmetry ambiguity (bottom row) between case 1 (yellow) and case 2 with reversed sequence (cyan). FIG. 14A panel B shows 4Pi-PSFs of channel p1 at z positions ranging from ±1 μm with vertical coma aberrations of −0.5 and 1.5λ/2π (case 3, top row) and −1.5 and 0.5 2/2π (case 4, middle row) in the upper and lower pupil functions, respectively. Cases 3 and 4 show identical PSFs at all Z positions. Overlapping 4Pi-PSFs with indistinguishable ambiguity (bottom row) between case 3 (yellow) and case 4 (cyan). FIG. 14B panel C shows Effect of covariant and contravariant objective misalignment in 4Pi-SMSN. FIG. 14B panel D shows 4Pi-PSFs of channel p1 at z positions ranging from +1 μm with vertical coma aberrations of −0 and 4λ/2π (case 5, top row) and −2 and 2λ/2π (case 6, middle row) in the upper and lower pupil functions, respectively. Cyan dashed line highlights the translation between identical PSF patterns in cases 5 and 6. Overlapping 4Pi-PSFs with indistinguishable translated ambiguity (bottom row) between case 5 (yellow) and case 6 with translation (cyan).

FIG. 15 panels A-L show demonstrations of spine morphologies as horizontal cross-sections (left), vertical cross-sections of spine neck (middle), and vertical cross-sections of spine head (right), color-coded according to single-molecule axial positions (n=12 spines in FIG. 6 panel J). Yellow curve is the trace of algorithm from manual identification.

FIG. 16 is a high-level diagram showing the components of an exemplary data-processing system.

### DETAILED DESCRIPTION

Single-molecule super-resolution microscopy allows pin-pointing individual molecular positions in cells with nanometer precision. However, achieving molecular resolution through tissues is often difficult because of optical scattering and aberrations. In certain aspects, the invention provides 4Pi single-molecule nanoscopy for for tissue, such as but not limited to brain, with in-situ point spread function retrieval through opaque tissue (4Pi-BRAINSPOT), integrating 4Pi single-molecule switching nanoscopy with dynamic in-situ coherent PSF modeling, single-molecule compatible tissue clearing, light-sheet illumination, and a novel quantitative analysis pipeline utilizing the highly accurate 3D molecular coordinates. This approach enables the quantification of protein distribution with sub-15-nm resolution in all three dimensions in complex tissue specimens. The invention herein has demonstrated 4Pi-BRAINSPOT's capacities in revealing the molecular arrangements in various sub-cellular organelles and resolved the membrane morphology of individual dendritic spines through 50-μm transgenic mouse brain slices. This ultra-high-resolution approach allows one to decipher nanoscale organelle architecture and molecular distribution in both isolated cells and native tissue environments with precision down to a few nanometers.

### Design of 4Pi-BRAINSPOT

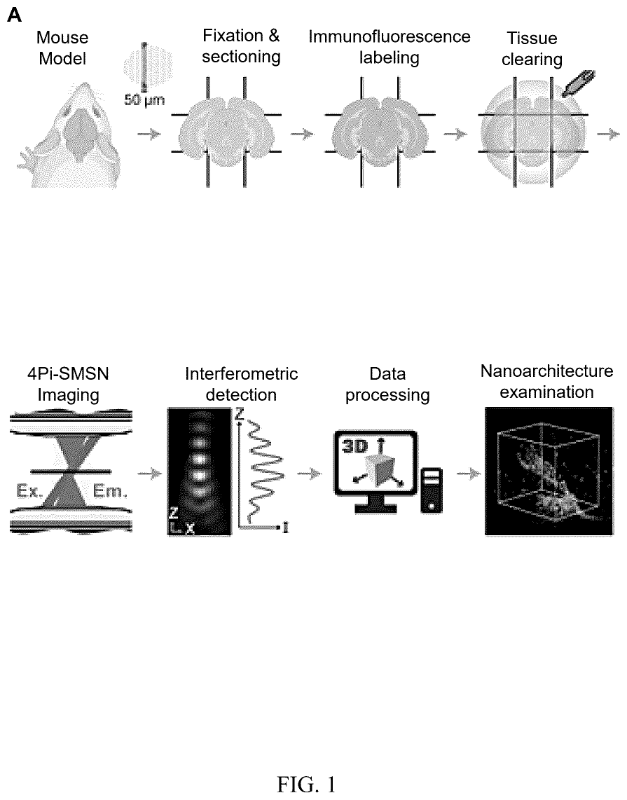

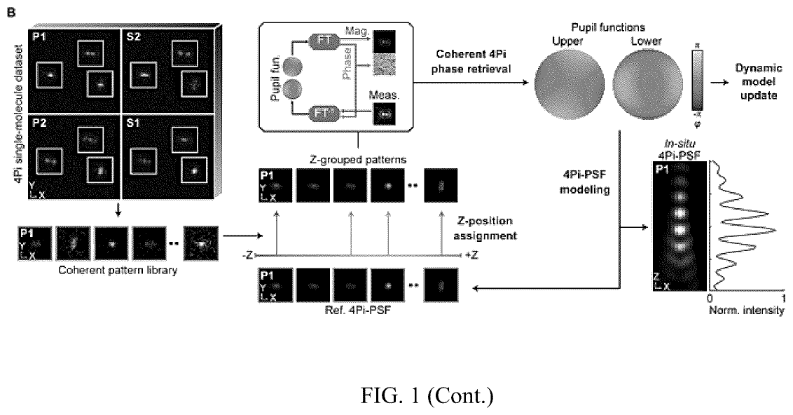

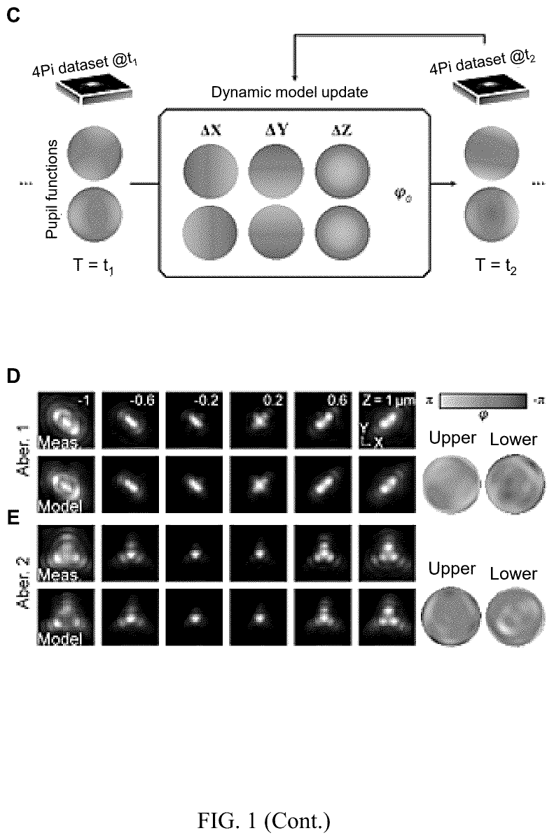

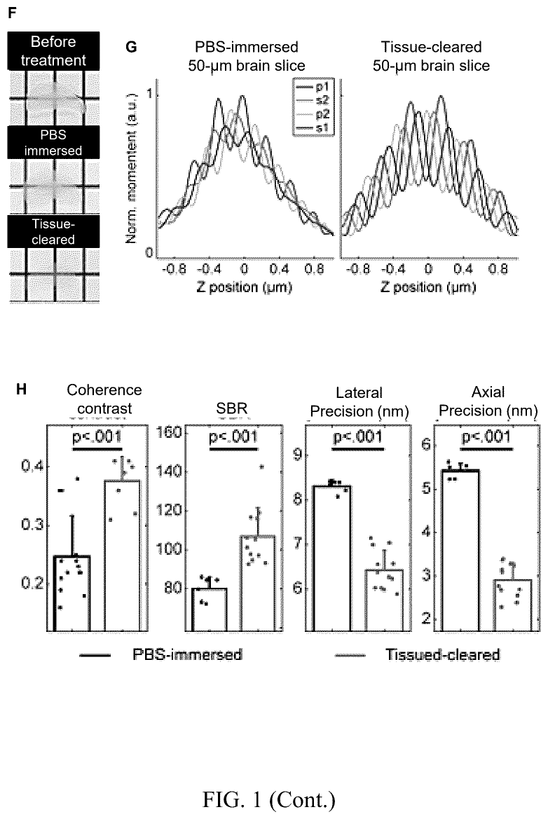

4Pi-BRAINSPOT (FIG. 1 panel A) is developed based on 4Pi-SMSN, which coherently detects the emitted photons from single emitters through two conjugated objectives achieving sub-10 nm localization precision in all three dimensions. However, 4Pi-SMSN through tissues remains difficult. Biological tissues have complex optical properties, causing scattering and aberrations and thus deteriorating the interferometric emission pattern. Additionally, temperature-induced changes in the coherent cavity and persistent objective misalignments also affect the achievable interferometric contrast and emission pattern of single molecules.

To mitigate tissue-induced aberration and temporal fluctuations in the interferometric system, the invention herein provides a 4Pi in-situ point spread function (PSF) retrieval algorithm, which enables direct retrieval of an aberrated, time-dependent in-situ model of the single-molecule emission patterns from the 4Pi-SMSN dataset captured in brain specimens (FIG. 1 panels B-C and Examples). By deriving the model in situ, it reflects the distortions, blurring, and partial coherence encountered during detection, and thereby minimizes disparities between the data and the model. This approach provides accurate molecular localizations in the presence of tissue-induced aberrations.

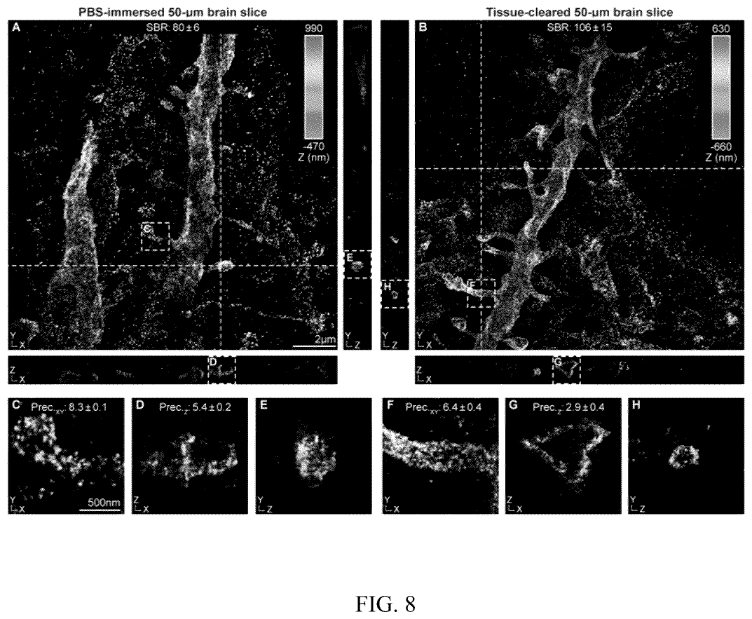

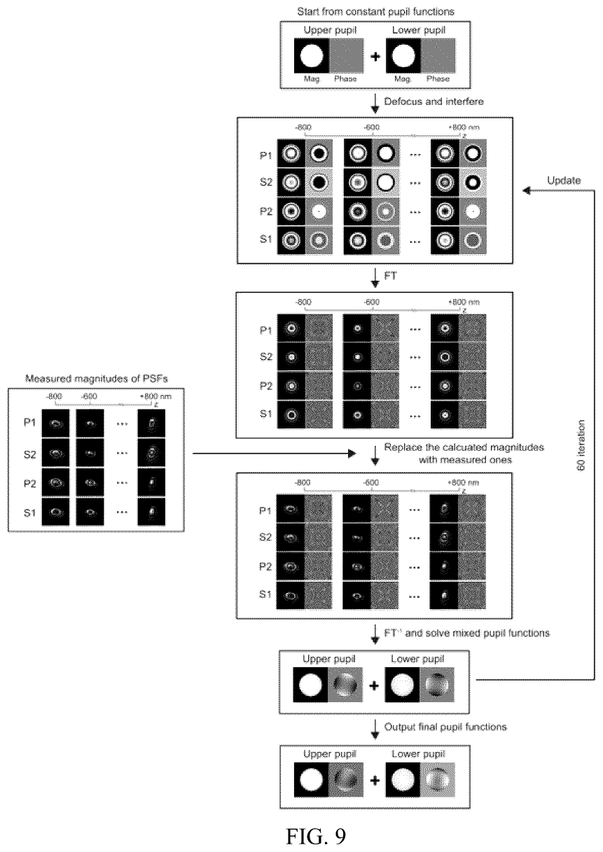

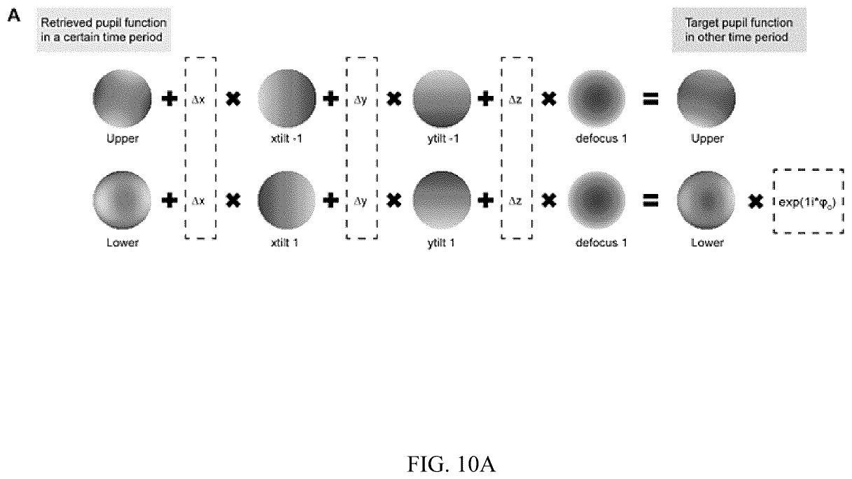

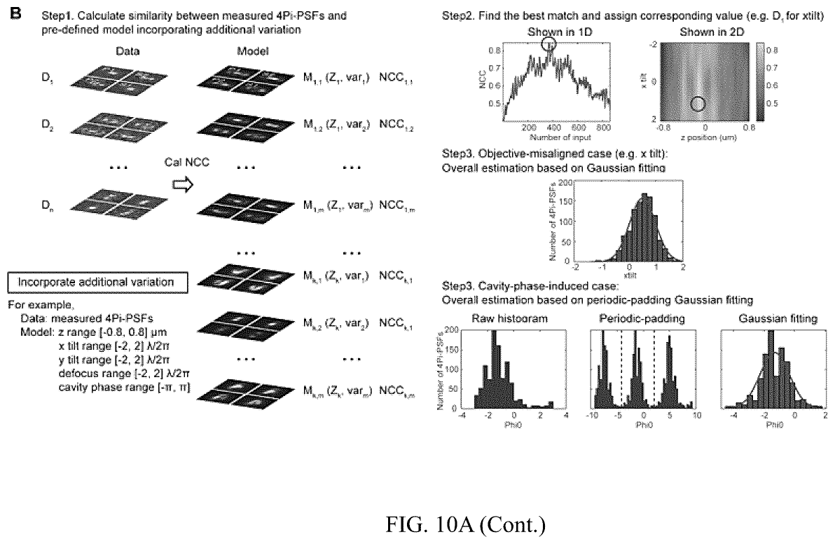

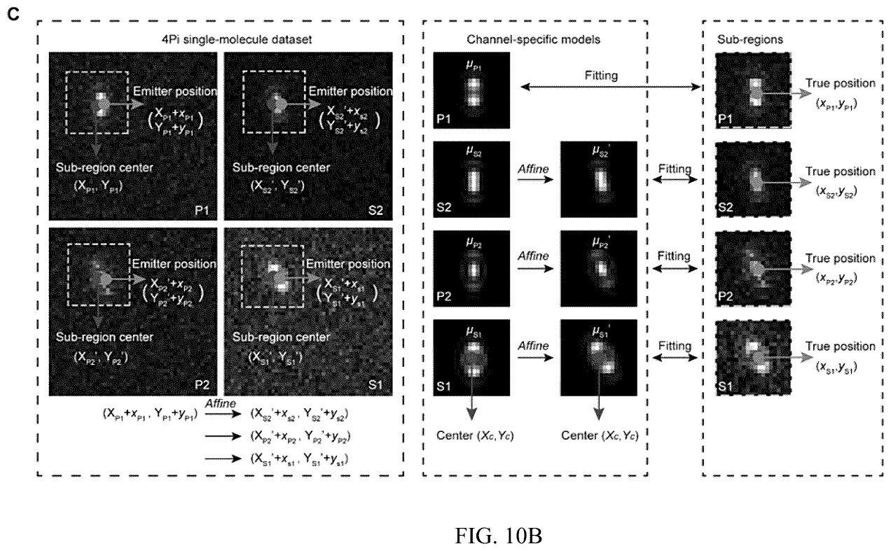

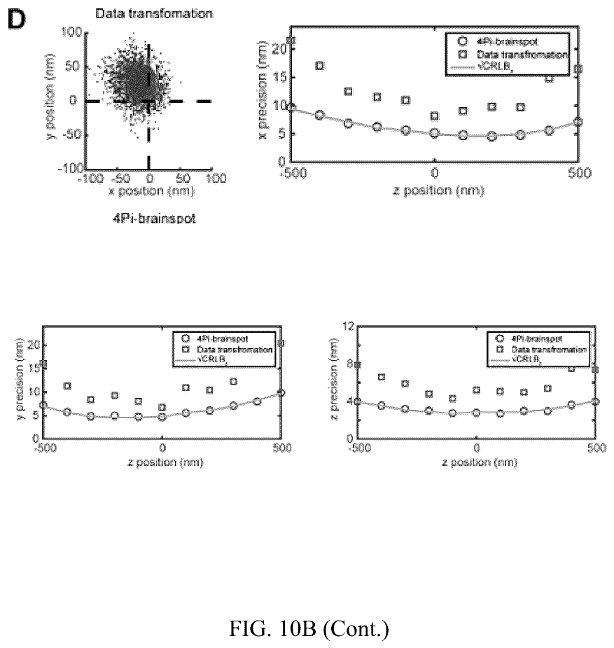

The 4Pi in-situ PSF retrieval algorithm begins by segmenting multichannel 4Pi emission patterns to create a library of detected emission patterns. These segments, from stochastically blinking events at varying axial positions, serve as random samples of the underlying in-situ interferometric (4Pi) PSF that we aim to retrieve. To estimate axial positions of patterns in the library, reference PSFs are generated from coherent pupil functions, depicting the interferometric amplitude and phase of wavefield superposition from the pupils of the two opposing objectives. The detected emission patterns are then assigned to specific axial groups according to their similarity to the reference PSFs. The categorized patterns are then aligned and averaged within each group to generate experimental PSF observations. To retrieve the individual pupil functions, we developed a coherent 4Pi phase retrieval algorithm based on the Gerchberg-Saxton algorithm. This allows us to retrieve individual pupil functions for each objective from their coherent superpositions and update the reference 4Pi-PSFs for the subsequent iteration (FIG. 9. Through sequential iterations, the model is refined to capture detailed coherent aberrations from both instrumental imperfections and biological specimens, representing in-situ interferometric emission patterns (FIG. 1 panels D-E) for accurate and precise single-molecule localization inside tissue specimens (FIG. 10B panel C).

To address temporal aberrations induced by the system and specimens, commonly encountered due to the extended acquisition time of 4Pi-SMSN, 4Pi-BRAINSPOT employs a dynamic in-situ model update to mitigate drift-induced disparities between the acquired 4Pi data and the 4Pi-PSF model. In brief, 4Pi-BRAINSPOT detects and iteratively updates the extracted coherent pupil functions to capture the temporal alterations of single-molecule patterns caused by time-dependent objective misalignments and interferometric cavity shifts (FIG. 10A panels A-B). Compared to traditional fiducial-bead-based in-vitro approaches, these dynamic refinements result in a time-varying response function of the 4Pi-SMSN unique to each time point.

4Pi-BRAINSPOT further incorporates an SMSN-compatible tissue-clearing method to mitigate aberration and scattering within opaque specimens, thereby preventing the permanent loss of Fisher information. We found that a combination of fast optical clearing method (FOCM) facilitated by protein denaturation and tissue hyperhydration, with a thiol-based imaging buffer significantly reduces tissue heterogeneity while preserving the photo-switching behavior of fluorophores critical for SMSN experiments (FIG. 1 panel F). Comparative results with and without FOCM inside 50-μm thick samples (FIG. 1 panels G-H) indicate a 1.5-fold increase in coherent fringe contrast (from 0.25±0.07, mean±SD, n=14 beads, to 0.38±0.04, n=8 beads) and a 1.3-fold increase in signal-to-background ratio (SBR, from 80.1±6.0, n=6 datasets, to 106.9±14.7, n=12 datasets). Furthermore, by introducing highly inclined and laminated optical sheet (HILO) as excitation46,4Pi-BRAINSPOT further enables a 60% reduction in out-of-focus background across 50-μm thick brain sections. These improvements collectively led to a localization precision of 6.4±0.4 nm laterally and 2.9±0.4 nm axially as quantified through tissue specimens (n=12 datasets; FIG. 8 panels A-H).

### Characterization of 4Pi-BRAINSPOT Performance

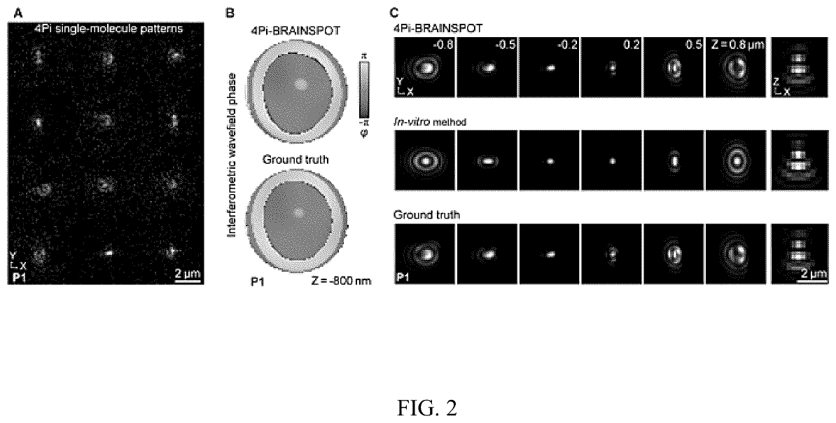

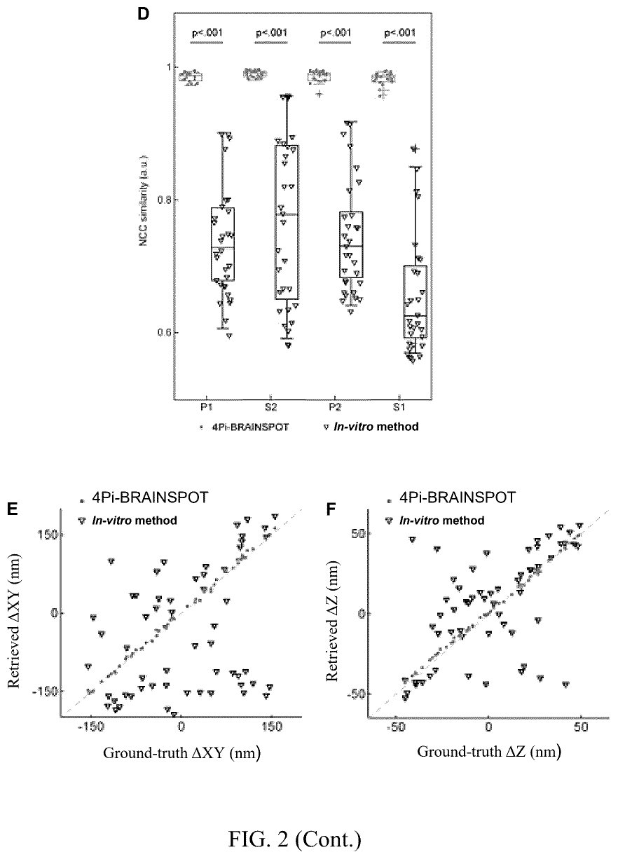

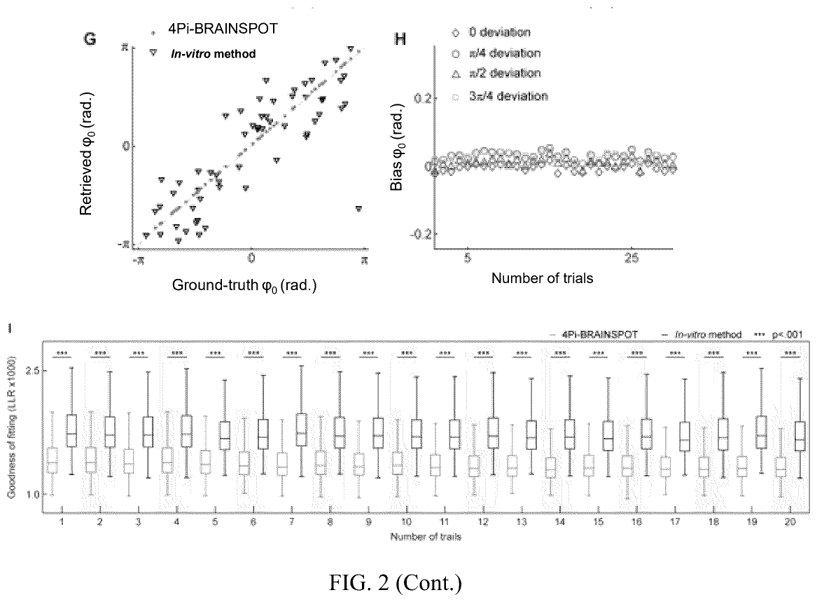

The accuracy and precision of in-situ PSF modeling was initial tested by retrieving known distortions from simulated 4Pi emission patterns with random axial positions in a range of ±800 nm (FIG. 2 panel A). The simulated aberrations included 11 Zernike modes with random amplitudes between +1λ/2π, with vertical astigmatism as a known prior breaking the axial ambiguity. The methodology herein successfully retrieved the intricate fringes of the interferometric patterns (FIG. 2 panels B-D), achieving a normalized cross-correlation similarity (NCC) of 0.98±0.05 (n=30 trials; Method 3.3) comparing to the ground truth, consistently outperforming conventional in-vitro approaches (NCC of 0.75±0.20, n=60 trials). Furthermore, we assessed the algorithm's ability to retrieve lateral and axial objective misalignments (ΔXY & AZ) and dynamic system shifts as cavity phase differences (φ0) (FIG. 2 panels E-G). Our approach allowed us to retrieve these commonly encountered interferometric system drifts at lateral and axial directions with standard deviations of 4.4 nm and 1.4 nm (n=60 trials) and captured the cavity phase difference consistently and accurately with a standard deviation of 12.7 mrad (n=60 trials). The algorithm also accurately captured the cavity phase with deviated initial guesses, indicating minimal influence of initial guess in obtaining an accurate PSF model (FIG. 2 panel H).

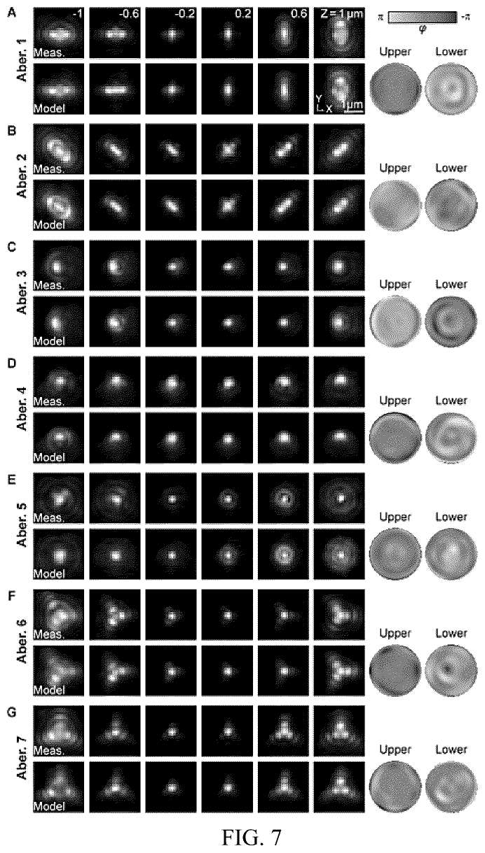

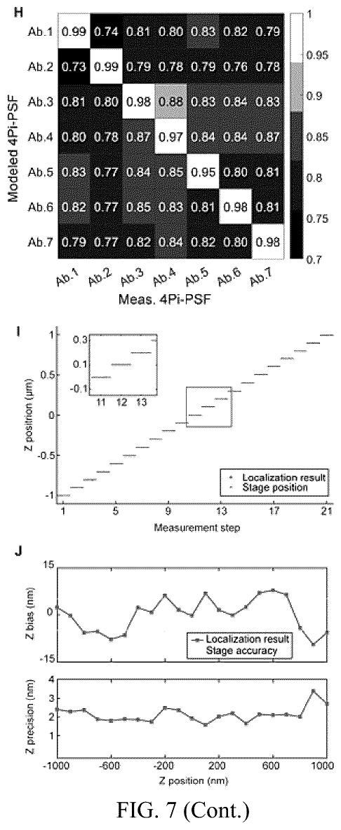

4Pi-BRAINSPOT was further evaluated with induced aberrations where 4Pi-BRAINSPOT is used to retrieve coherent PSF models from the measured data where independent wavefront distortions are generated by deformable mirrors in both interferometric arms. Among all 7 types of commonly encountered aberrations, represented by Zernike polynomials (FIG. 7 panels A-H), in-situ 4Pi-PSF models achieved high similarity compared to the recorded ones (NCC of 0.98±0.01, n=14 trials), a significant improvement over their in-vitro or theoretical counterparts (NCC of 0.81±0.04, n=84 trials). Our method also achieved an axial precision of 2-3 nm in a range of ±800 nm when localizing fluorescent beads, demonstrating the stability of the interferometric system (FIG. 7 panels I-J). Next, we assessed the capability of 4Pi-BRAINSPOT algorithm to capture dynamic 4Pi distortions while imaging immunofluorescence-labeled TOM20 in U2OS cells. To characterize the reliability of the time-dependent model update, we introduced random aberrations via deformable mirrors at various time points during imaging. We evaluated the goodness of fitting between the captured emission patterns in cells and the in situ retrieved PSF models by calculating the log-likelihood ratio (LLR) (FIG. 2 panel I). The methods herein demonstrated an LLR of 1384±208 across 20 trials with various aberrations, consistently outperforming the conventional static in-situ model constructed from bead measurement near the cells with an LLR of 1769±298 (n=200,218 localizations).

### Nanoscale Visualization and Quantitative Analysis of Subcellular Organisms in Mammalian Cells

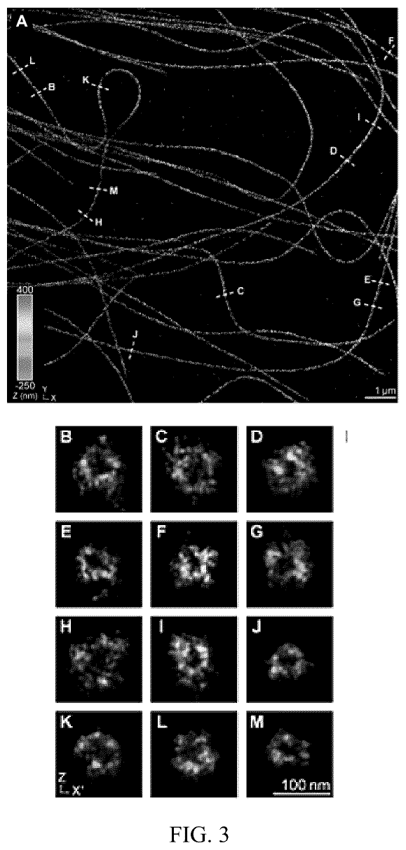

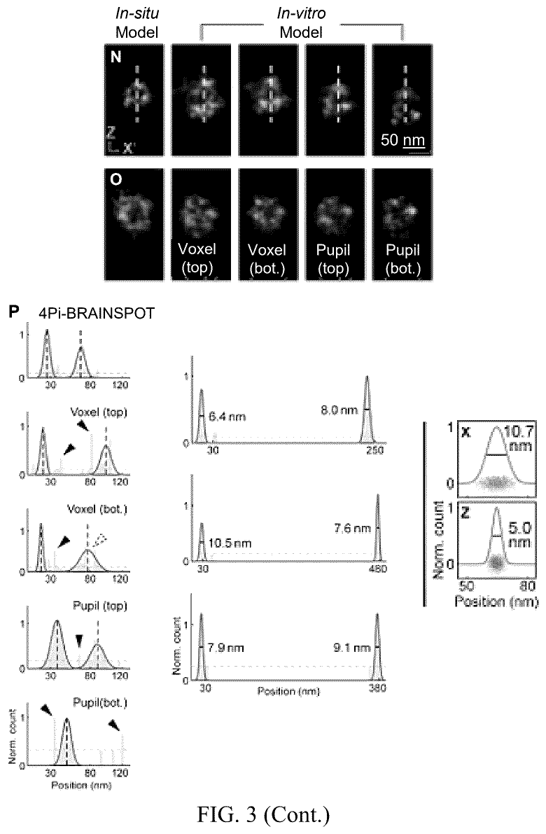

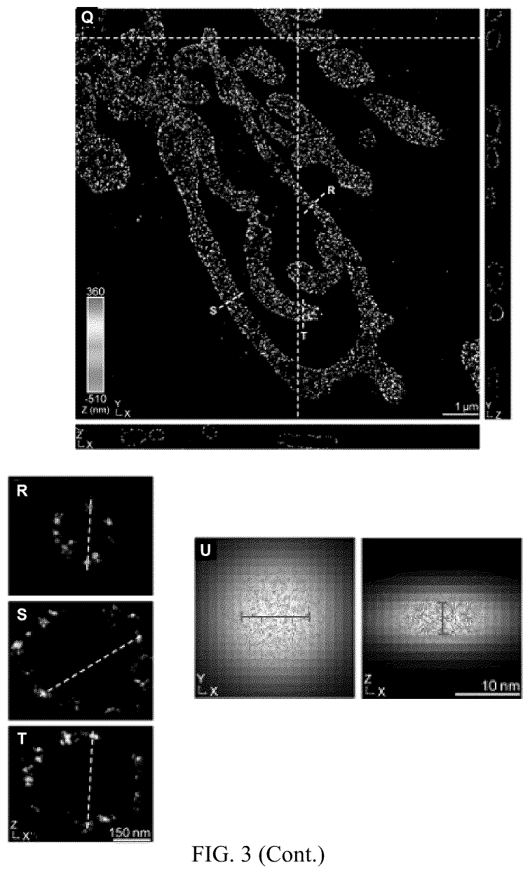

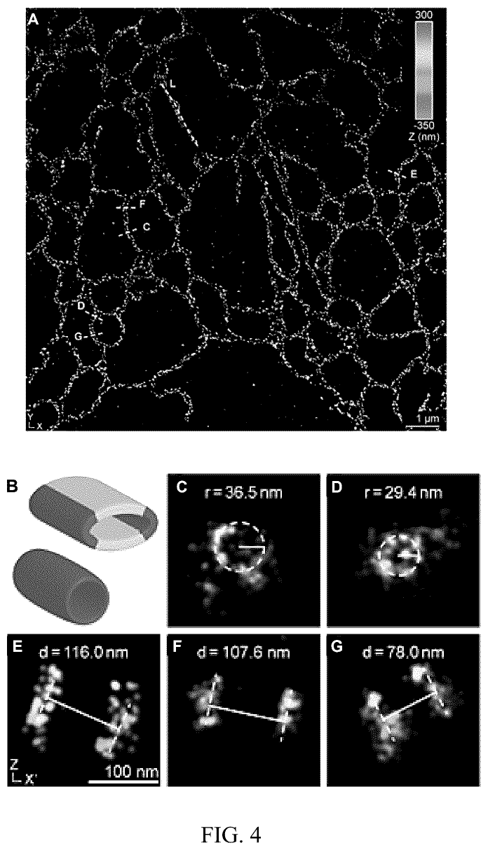

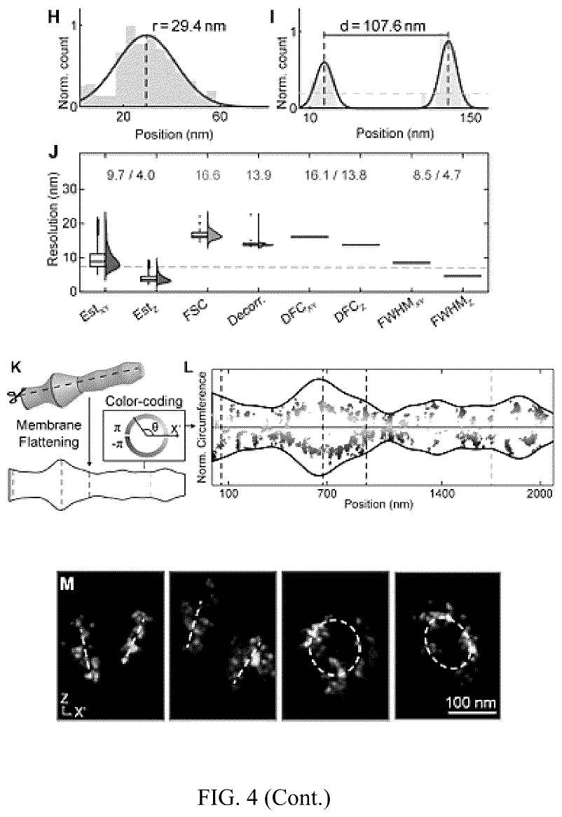

To demonstrate the capabilities of 4Pi-BRAINSPOT in resolving subcellular ultrastructure, we imaged microtubular structure immunofluorescence-labeled with α-tubulin in COS-7 cells, known to be 3D tubules with diameters of 50-70 nm39,47,48, on top coverslips with a 25-μm cavity, as a controlled aberrated condition (FIG. 4 panel A). The inhomogeneous refractive indices of the specimen, water-based mounting medium, and objective immersion oil cause refractive index mismatch aberrations and a random change in the cavity phase. These, in turn, exacerbate errors when pin-pointing the center localizations of single emitters through thick specimens due to the mismatch between the captured emission pattern and the in-vitro bead model. We compared the result of the in-situ model obtained from 4Pi-BRAINSPOT with that of two in-vitro modeling methods-voxel-based and pupil-based modeling—using bead measurements taken from both the top coverslip near the structures of interest and the bottom coverslip away from the structures (FIG. 3 panels N—O). With 4Pi-BRAINSPOT, we found that the expected hollow tubular structures of microtubules could be revealed as distinct ring cross-sections at various positions and depths (FIG. 3 panels B-M). In comparison, the result of 4Pi-BRAINSPOT illustrated sharp contours, whereas the other methods exhibited loose distributions or high localization variability (FIG. 3 panel P).

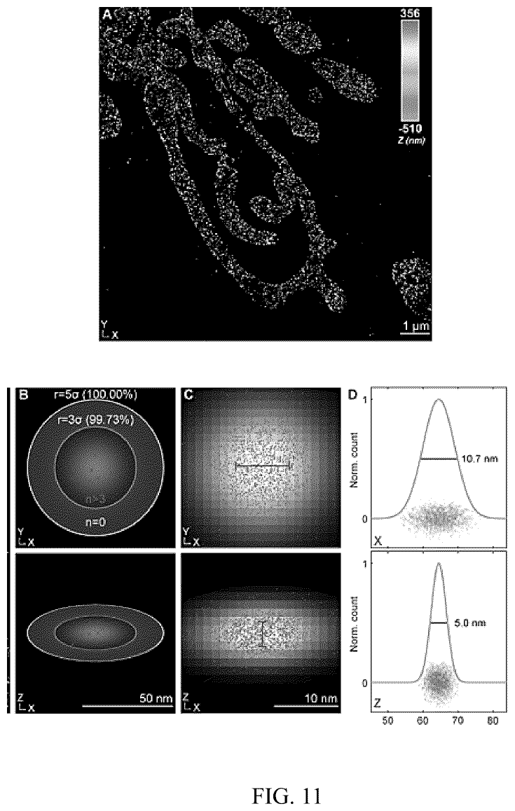

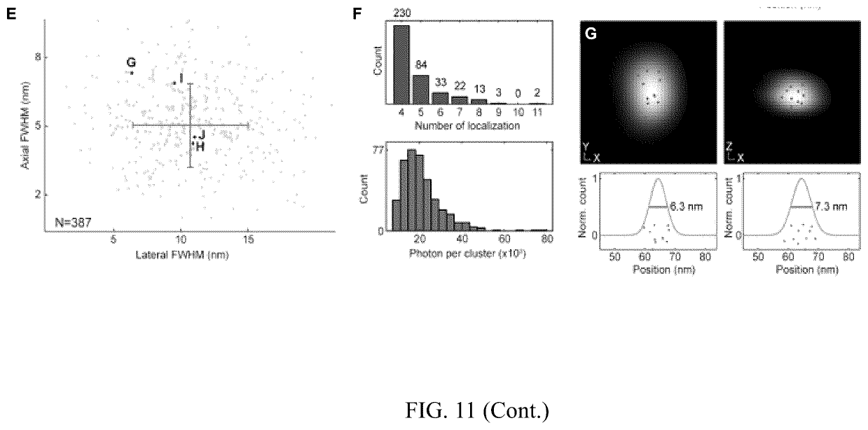

Next, we imaged mitochondria immunofluorescence-labeled with TOM20 in COS-7 cells, a receptor protein clustering on the outer mitochondrial membrane49-51, through a 25 μm water-based cavity (FIG. 3 panel Q). We successfully visualized the nanoscale distribution of the TOM20 clusters on the mitochondrial surface, achieving a full-width half maximum (FWHM) of 6-10 nm (FIG. 3 panels R-T). To validate the achieved molecular localization precision in situ, we measured the sizes of localization distribution formed by overlapping 3D-aligned single-molecule localization clusters (FIG. 11 panels A-J). These measurements aligned with the estimated resolution of 11.3±3.8 nm laterally and 4.7±1.5 nm axially (corresponding to Cramér-Rao lower bound localization precision of 4.8±1.6 nm and 2.0±0.6 nm, n=1845 localizations across 387 clusters).

Furthermore, we examined the organization of immunofluorescence-labeled Reticulon 4 (Rtn4), a protein stabilizing the curvature of the endoplasmic reticulum (ER) membrane, in COS-7 cells (FIG. 4 panel A). Despite various hypotheses about Rtn4's role were suggested, traditional microscopy has been limited in providing nanoscale 3D insights into Rtn4 regulation in the ring closure process. Using 4Pi-BRAINSPOT, we observed two distinct patterns of Rtn4-enriched ER membranes (FIG. 4 panel B); narrow-waist ring structures about 30-nm radius (FIG. 4 panels C-D and H), indicative of ER tubules with small round lumens, and parallel lines separated by 100 nm (FIG. 4 panels E-G and I), suggesting ER tubules with larger elliptical lumens. The result consisting of high-precision molecular localizations in 3D allowed us to numerically flatten the ER membrane, map the angular distribution of Rtn4 clusters, and visualize the evolving spatial arrangement and transition of Rtn4 along an isolated ER segment (FIG. 4 panels K-M).

4Pi-BRAINSPOT resolves ultrastructure of dendrites and spines through brain tissues Dendritic spines are crucial for synaptic connections and play a key role in memory processing by reflecting the brain's adaptive capacity through their morphology changes driven by synaptic plasticity. Synaptic potentiation can prompt the formation of new spines or alter the concavity of the spine head and the dimensions of the spine neck. Thin spines are often linked to learning and memory, mushroom-shaped spines to mature synaptic connections, and stubby spines to developmental or repair processes. Furthermore, irregularities in spine organization and density are also associated with synaptic connectivity disruptions. Understanding these nanoscale changes in spines is crucial for elucidating the mechanisms of synaptic connection and pathology of neurological disorders.

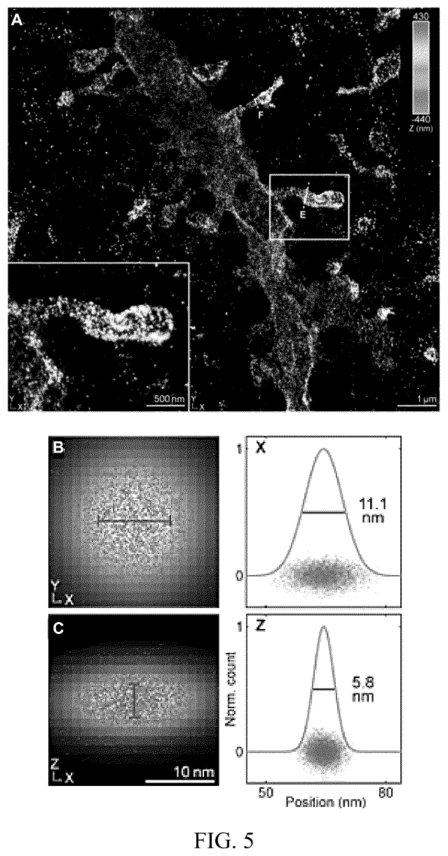

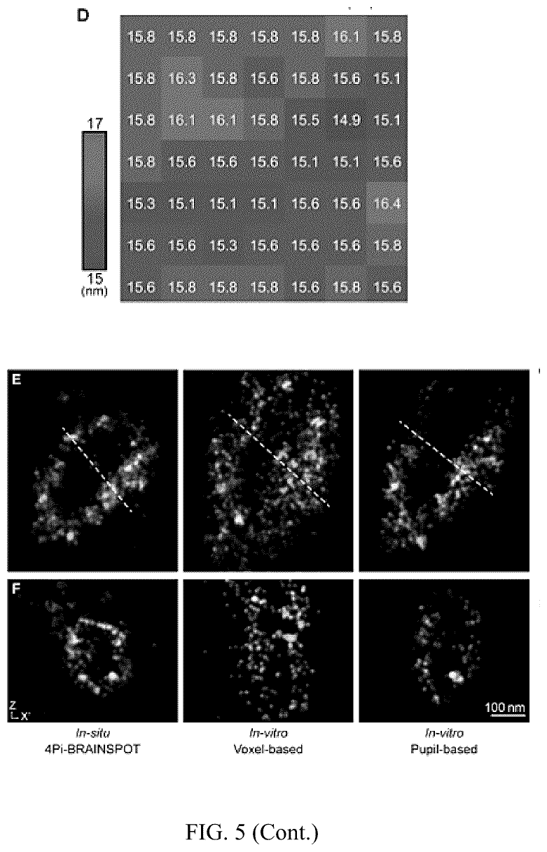

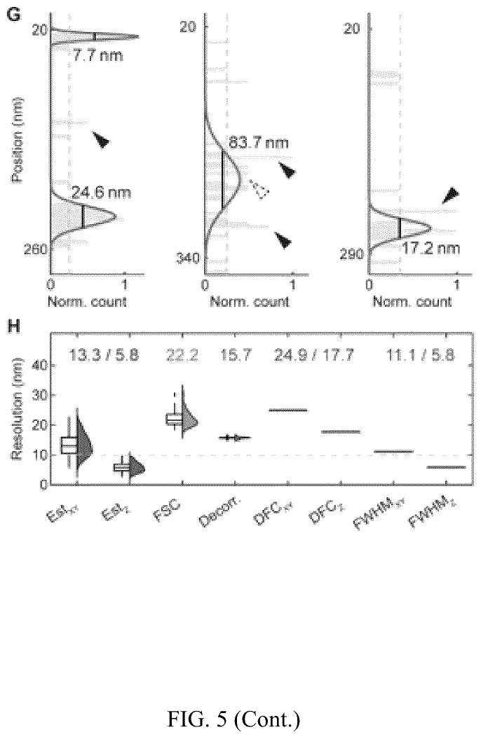

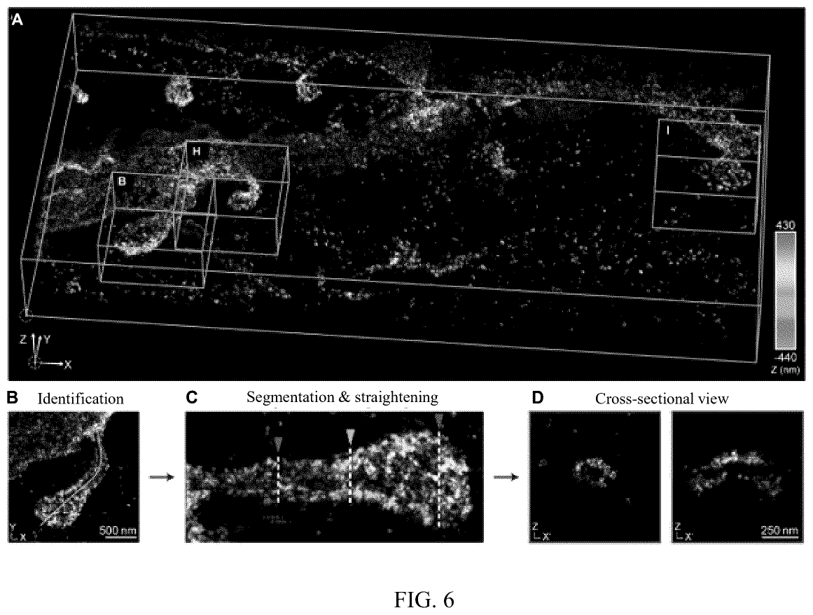

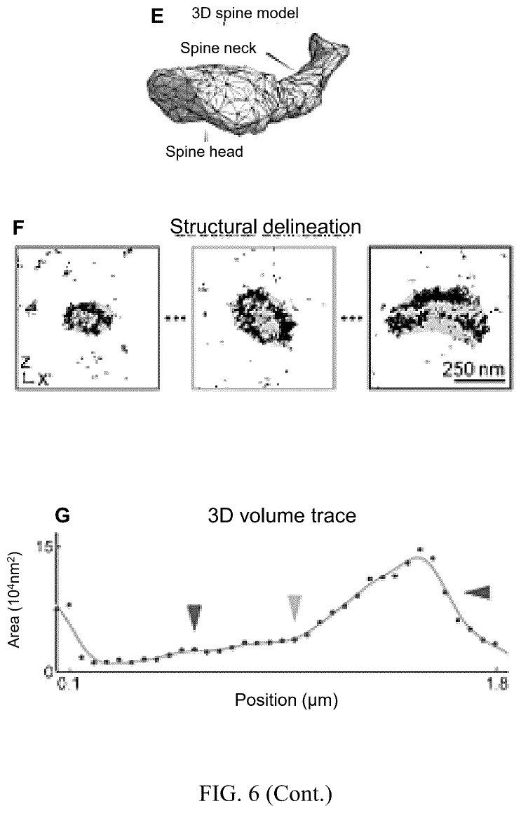

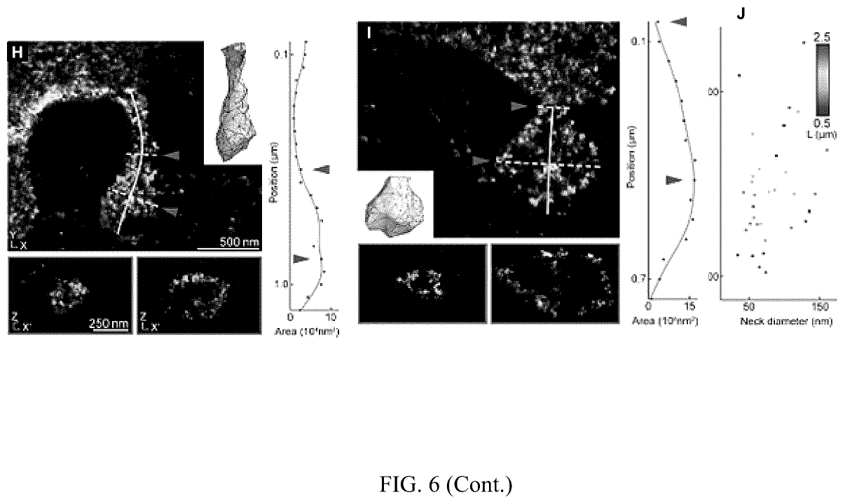

Channelrhodopsin-2 (ChR2), a light-gated ion channel, serves as an important role for investigating optogenetic modulation. Its distribution and clustering within the neuronal membrane makes it an ideal target for assessing the imaging fidelity of our method. To visualize the spatial distribution of these ion channels, we imaged 50-μm mouse brain slices and successfully located the distinct membrane-associated protein clusters throughout dendritic spines. Our quantitative resolution analysis indicated an achieved sub-15 nm resolution, and the resulting reconstruction revealed the 3D molecular distribution of ChR2 on dendritic spines (FIG. 5 panel A and FIG. 6 panel A). We were able to observe structural transitions from dendritic shafts to spine heads, where the 3D localized ChR2 molecular cloud provided detailed morphological information. We also observed a distinct concave spine head on a long and mushroom-shaped spine, potentially indicating an increased postsynaptic density area in response to synaptic adaptation.

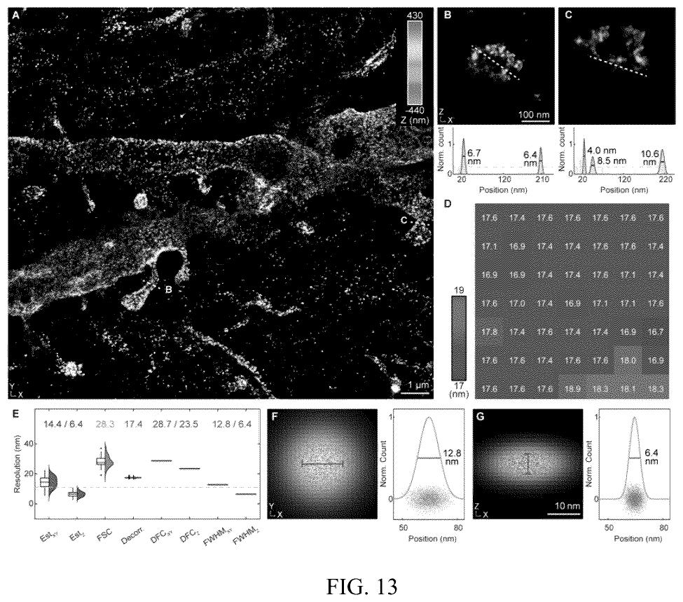

To assess the performance of 4Pi-BRAINSPOT through brain tissues, we evaluated the resolution with multiple criteria. Our cluster analysis pipeline indicated that the overall size of single-molecule clusters distributed through different tissues was 13.0±2.2 nm laterally and 6.8±1.3 axially (n=7 datasets; FIG. 5 panels B-C, FIG. 13 panels F-G). We verified it by measuring the FWHM of the neuronal membrane waist as ˜8 nm (FIG. 5 panel G, FIG. 13 panels B-C). These measurements are consistent with the estimated resolution of 15.1±2.6 nm laterally and 7.0±1.6 axially. To evaluate the achievable resolution under certain labeling density and imaging conditions, we utilized directional Fourier correlation (DFC) analysis to calculate Fourier correlation resolution in spatial-frequency domains. The DFC results indicated lateral and axial resolutions of 30.3±5.8 nm and 21.0±4.7 nm, closely matching the results from 2D imaging decorrelation analysis of 17.8±3.5 nm (FIG. 5 panel D, FIG. 13 panel D) and 3D Fourier shell correlation of 26.6±5.0 nm (n=7 datasets; FIG. 5 panel H, FIG. 13 panels E).

Furthermore, we developed a tailored pipeline for processing 4Pi-SMSN data to quantitatively analyze dendritic spine morphology based on the resulting molecular coordinates of individual membrane-bounded proteins in 3D (FIG. 6 panels B-G). This method, first, segments single molecule localizations into sequential cross-sections, then detects the complex boundaries of major structures from scattered molecular centers using the alpha-shape algorithm, which unites multiple convex hulls encompassing localization subsets and finally pinpoints structural centers by fitting ellipses to localizations within the alpha-shaped boundaries. The radii of the isolated spine sections are then estimated by calculating the mode of distances between the center and the localizations. Using the automated quantification pipeline, we analyzed the ChR2-outlined membrane contours and quantified the topographic and volumetric changes of spines (FIG. 6 panels H-I). In a collection of 33 identified spines imaged at the visual cortex resolved from brain tissues (FIG. 6 panel J), we observed a diverse range of spine morphologies, from 0.6-μm-long round-shaped spines to 2.4-μm-long mushroom-shaped spines. The spine neck radii varied from 34.1 to 159.7 nm (81.1±33.6 nm), and spine heads radii varied from 104.3 to 353.3 nm (193.1±59.3 nm), with total volumes ranging from 0.02 to 0.36 μm3 (0.10±0.07 μm3), highlighting the effectiveness of our method in analyzing spines with diverse arrangements and orientations (FIG. 15 panels A-L).

### Additional Embodiments

Understanding the brain has long been one of the most ambitious and compelling endeavors in science. However, a grand challenge remains in neuroscience—visualizing how sensory experiences sculpt neural circuits and synaptic connections at the subcellular level has been limited due to the challenges of achieving sufficient resolution and molecular specificity deep within the brain. While previous research has made significant contributions to understanding the molecular and cellular mechanisms underlying neural plasticity, current super-resolution imaging techniques have mostly been limited to cultured preparations. Despite 2-photon imaging being widely used in live animals, its intrinsic diffraction limit prevents it from capturing fine structures and protein localizations deep within neural tissues70,71. 4Pi-BRAINSPOT bridges this profound gap by stepping forward in achievable resolution and depth, enabling deciphering nanoscale molecular architecture deep within the brain tissues with unparalleled clarity. Furthermore, the highly accurate 3D molecular map generated by 4Pi-BRAINSPOT supports a novel analysis pipeline that identifies, segments, and quantifies the morphological properties of dendritic spines. This capability facilitates advanced studies of synaptic architecture and plasticity, fundamental processes underlying learning, memory, and information processing.

Further enhancement for the 4Pi-BRAINSPOT system involves increasing throughput volume and addressing potential field-dependent distortions. When imaging a large FOV, these lateral-dependent distortions may alter the 4Pi-PSF, and thus challenge the assumption of shift-invariant PSF72,73. To mitigate these issues, one might integrate field lenses specifically designed to compensate for FOV-dependent distortions at the instrumental level and develop algorithms to adapt to these variations. Implementing these improvements will ensure that in-situ modeling accuracy is maintained across the extended FOVs, and thus its localization performance for high-fidelity, high-throughput imaging applications. Integrating functional imaging techniques will also enhance 4Pi-BRAINSPOT's ability to elucidate the molecular mechanisms underlying neural activities 30,32. Utilizing 4Pi-BRAINSPOT in thick brain tissue to study the perceptual experience-dependent plasticity of dendritic spines can correlate with the functional synaptic measurements by electrophysiology experiment. This would enable nanoscale molecular exploration, providing a comprehensive understanding of how structural changes at the nanoscale level impact overall neuronal function and behavior. We believe 4Pi-BRAINSPOT marks a substantial advancement in advanced optical nanoscopy, offering unprecedented insights into the nanoscale organization of biological tissues. We hope this unique observation capacity near the molecular level will further our understanding of cellular functions, neural circuits, and disease pathologies.

### System Architecture

FIG. XX is a high-level diagram showing the components of an exemplary data-processing system 1000 for analyzing data and performing other analyses described herein, and related components. The system includes a processor 1086, a peripheral system 1020, a user interface system 1030, and a data storage system 1040. The peripheral system 1020, the user interface system 1030 and the data storage system 1040 are communicatively connected to the processor 1086. Processor 1086 can be communicatively connected to network 1050 (shown in phantom), e.g., the Internet or a leased line, as discussed below. The data described above may be obtained using detector 1021 and/or displayed using display units (included in user interface system 1030) which can each include one or more of systems 1086, 1020, 1030, 1040, and can each connect to one or more network(s) 1050. Processor 1086, and other processing devices described herein, can each include one or more microprocessors, microcontrollers, field-programmable gate arrays (FPGAs), application-specific integrated circuits (ASICs), programmable logic devices (PLDs), programmable logic arrays (PLAs), programmable array logic devices (PALs), or digital signal processors (DSPs).

Processor 1086 which in one embodiment may be capable of real-time calculations (and in an alternative embodiment configured to perform calculations on a non-real-time basis and store the results of calculations for use later) can implement processes of various aspects described herein. Processor 1086 can be or include one or more device(s) for automatically operating on data, e.g., a central processing unit (CPU), microcontroller (MCU), desktop computer, laptop computer, mainframe computer, personal digital assistant, digital camera, cellular phone, smartphone, or any other device for processing data, managing data, or handling data, whether implemented with electrical, magnetic, optical, biological components, or otherwise. The phrase “communicatively connected” includes any type of connection, wired or wireless, for communicating data between devices or processors. These devices or processors can be located in physical proximity or not. For example, subsystems such as peripheral system 1020, user interface system 1030, and data storage system 1040 are shown separately from the data processing system 1086 but can be stored completely or partially within the data processing system 1086.

The peripheral system 1020 can include one or more devices configured to provide digital content records to the processor 1086. For example, the peripheral system 1020 can include medical devices (such as medical imaging devices), digital still cameras, digital video cameras, cellular phones, or other data processors. The processor 1086, upon receipt of digital content records from a device in the peripheral system 1020, can store such digital content records in the data storage system 1040.

The user interface system 1030 can include a mouse, a keyboard, another computer (e.g., a tablet) connected, e.g., via a network or a null-modem cable, or any device or combination of devices from which data is input to the processor 1086. The user interface system 1030 also can include a display device, a processor-accessible memory, or any device or combination of devices to which data is output by the processor 1086. The user interface system 1030 and the data storage system 1040 can share a processor-accessible memory.

In various aspects, processor 1086 includes or is connected to communication interface 1015 that is coupled via network link 1016 (shown in phantom) to network 1050. For example, communication interface 1015 can include an integrated services digital network (ISDN) terminal adapter or a modem to communicate data via a telephone line; a network interface to communicate data via a local-area network (LAN), e.g., an Ethernet LAN, or wide-area network (WAN); or a radio to communicate data via a wireless link, e.g., WiFi or GSM. Communication interface 1015 sends and receives electrical, electromagnetic or optical signals that carry digital or analog data streams representing various types of information across network link 1016 to network 1050. Network link 1016 can be connected to network 1050 via a switch, gateway, hub, router, or other networking device.

Processor 1086 can send messages and receive data, including program code, through network 1050, network link 1016 and communication interface 1015. For example, a server can store requested code for an application program (e.g., a JAVA applet) on a tangible non-volatile computer-readable storage medium to which it is connected. The server can retrieve the code from the medium and transmit it through network 1050 to communication interface 1015. The received code can be executed by processor 1086 as it is received, or stored in data storage system 1040 for later execution.

Data storage system 1040 can include or be communicatively connected with one or more processor-accessible memories configured to store information. The memories can be, e.g., within a chassis or as parts of a distributed system. The phrase “processor-accessible memory” is intended to include any data storage device to or from which processor 1086 can transfer data (using appropriate components of peripheral system 1020), whether volatile or nonvolatile; removable or fixed; electronic, magnetic, optical, chemical, mechanical, or otherwise. Exemplary processor-accessible memories include but are not limited to: registers, floppy disks, hard disks, tapes, bar codes, Compact Discs, DVDs, read-only memories (ROM), Universal Serial Bus (USB) interface memory device, erasable programmable read-only memories (EPROM, EEPROM, or Flash), remotely accessible hard drives, and random-access memories (RAMs). One of the processor-accessible memories in the data storage system 1040 can be a tangible non-transitory computer-readable storage medium, i.e., a non-transitory device or article of manufacture that participates in storing instructions that can be provided to processor 1086 for execution.

In an example, data storage system 1040 includes code memory 1041, e.g., a RAM, and disk 1043, e.g., a tangible computer-readable rotational storage device such as a hard drive. Computer program instructions are read into code memory 1041 from disk 1043. Processor 1086 then executes one or more sequences of the computer program instructions loaded into code memory 1041, as a result performing process steps described herein. In this way, processor 1086 carries out a computer implemented process. For example, steps of methods described herein, blocks of the flowchart illustrations or block diagrams herein, and combinations of those, can be implemented by computer program instructions. Code memory 1041 can also store data, or can store only code.

Various aspects described herein may be embodied as systems or methods. Accordingly, various aspects herein may take the form of an entirely hardware aspect, an entirely software aspect (including firmware, resident software, micro-code, etc.), or an aspect combining software and hardware aspects. These aspects can all generally be referred to herein as a “service,” “circuit,” “circuitry,” “module,” or “system.”

Furthermore, various aspects herein may be embodied as computer program products including computer readable program code stored on a tangible non-transitory computer readable medium. Such a medium can be manufactured as is conventional for such articles, e.g., by pressing a CD-ROM. The program code includes computer program instructions that can be loaded into processor 1086 (and possibly also other processors) to cause functions, acts, or operational steps of various aspects herein to be performed by the processor 1086 (or other processor). Computer program code for carrying out operations for various aspects described herein may be written in any combination of one or more programming language(s), and can be loaded from disk 1043 into code memory 1041 for execution. The program code may execute, e.g., entirely on processor 1086, partly on processor 1086 and partly on a remote computer connected to network 1050, or entirely on the remote computer.

### INCORPORATION BY REFERENCE

References and citations to other documents, such as patents, patent applications, patent publications, journals, books, papers, web contents, have been made throughout this disclosure, including to the Supplementary. The Supplementary, and all other such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

### EXAMPLES

### Example 1: Sample Preparation and Data Acquisition

### Preparation and Clearing of Coverslip

High-precision glass coverslips (25-mm diameter, CG15XH, Thorlabs) were cleaned through a series of steps to ensure their readiness for experimental use. The coverslips were first sonicated for 15 mins in a 1M KOH solution using an ultrasonic cleaner (M2800H, Branson). This was followed by a 15-min sonication in double-distilled water and another 15-min sonication in 70% ethanol. Between each sonication step, the coverslips were rinsed with double-distilled water to remove any residual liquid from their surfaces. After the sonication steps, the coverslips were placed in a 110° C. oven (89508-424, Avantor) and heated until they were completely dry.

### Sample Preparation of Fluorescent Bead on Coverslip

The cleaned coverslips were coated with poly-L-lysine solution (P4707, Sigma-Aldrich) by immersing them for 30 mins at room temperature (RT) and rinsed once with double-distilled water. The coverslips were then incubated with 100 μL of a 1:106 diluted solution of 100-nm crimson beads (100 nm crimson beads, Invitrogen) in double-distilled water for 30 mins at RT, followed by another rinse with double-distilled water. The samples were mounted by adding 50 μL of 97% 2,2′-thiodiethanol (TDE, 166782, Sigma-Aldrich) in double-distilled water between the treated coverslips and cleared coverslips. The sandwiched samples were sealed using silicone dental glue (Twinsil speed 22, Picodent) and left to dry at RT for 30 mins prior to imaging.

### Sample Preparation of Fluorescent Dye on Coverslip

The cleaned coverslips were immersed with poly-L-lysine solution (P4707, Sigma-Aldrich) for 30 mins at room temperature (RT) and rinsed once with double-distilled water. The coverslips were incubated with 100 μL of a 1:103 diluted Alexa 647 (A21236, Molecular Probes) in 0.1M NaHCO₃ for 30 mins at RT, followed by two rinses with 0.1M NaHCO₃. The samples were mounted by adding 50 μL of a mixture containing 2.5 mM 3,4-dihydroxybenzoic acid (PCA, 37580, Sigma-Aldrich), and 50 nM protocatechuate 3,4-dioxygenase (PCD, P8279-25UN, Sigma-Aldrich) in dSTORM buffer (10% wt/vol glucose in 50 mM Tris, 50 mM NaCl, pH 8.0) between the treated coverslips and the other cleared coverslips. The sandwiched samples were sealed using silicone dental glue and left to dry at RT for 30 mins prior to imaging.

### Cell Culture

COS-7 cells (CRL-1651, ATCC) were grown on clean coverslips placed in six-well plates. Before adding cells, the six-well plates were exposed to UV light for 1 hr. The cells were cultured in Dulbecco's modified eagle medium (DMEM, 30-2002, ATCC) supplemented with 10% v/v fetal bovine serum (FBS, A5670701, Gibco) and 100 U/mL penicillin-streptomycin (P-S, 15070063, Gibco). The culture conditions were maintained at 37° C. with 5% CO2 in a humidified incubator until the confluence of the cells reached approximately 60%. The cells were grown on coverslips for a duration ranging from 12 to 24 hours before fixation. U2OS Cells (300444, Cytion) were grown on clean coverslips placed in six-well plates. Before adding cells, the six-well plates were exposed to UV light for 1 hr. The cells were cultured in McCoy's 5A (16600, Gibco) supplemented with 10% v/v fetal bovine serum (FBS, A5670701, Gibco) and 100 U/mL penicillin-streptomycin. The culture conditions were maintained at 37° C. with 5% CO2 in a humidified incubator until the confluence of the cells reached approximately 60%. The cells were grown on coverslips for a duration ranging from 12 to 24 hours before fixation.

### Fixation and Immunofluorescence Labeling of Cells

The cell fixations and immunolabeling were tailored for different cellular structures. For immunofluorescence-labeled TOM20, cells were fixed with pre-warmed (37° C.) 4% paraformaldehyde (PFA, 1:4-diluted 16% paraformaldehyde solution, 15710, Electron Microscopy Sciences) in 1×PBS at RT for 15 mins with gentle rocking, followed by rinsing twice with 1× PBS. Autofluorescence quenching was performed with fresh 0.1% sodium borohydride (NaBH4, 452882, Sigma-Aldrich) in 1×PBS at RT for 7 minutes with gentle rocking, followed by three rinses with 1× PBS. Cell permeabilization was carried out using block buffer (3% bovine serum albumin (BSA) and 0.2% Triton X-100 in 1× PBS) at RT for 30 mins with gentle rocking, after which the solution was aspirated. The cells were incubated with primary antibodies (1:2000-diluted rabbit anti-TOM20 primary antibody (ab78547, Abcam) in antibody dilution buffer (1% BSA and 0.2% Triton X-100 in 1× PBS)) at 4° C. overnight and rinsed three times for 5 mins with wash buffer (0.05% Triton X-100 in 1×PBS). The cells were then incubated with secondary antibodies (1:500-diluted goat anti-rabbit AF647 secondary antibody (A21236, Life Technologies) in antibody dilution buffer) at 4° C. for 4 hours and rinsed three times for 5 mins with wash buffer. Post-fixation was performed with the same buffer as fixation at RT for 10 mins with gentle rocking, followed by three rinses with 1×PBS. The cells were stored in 1× PBS at 4° C. until imaging.

For immunofluorescence-labeled α-tubulin, cells were pre-extracted with pre-warmed 0.2% saponin (SAE0073, Sigma-Aldrich) in cytoskeleton buffer (CBS, 10 mM MES pH 6.0, 138 mM NaCl, 3 mM MgCl2, 2 mM EGTA, 320 mM sucrose) at RT for 1 min with gentle rocking, after which the solution was aspirated. Subsequently, the cells were fixed with pre-warmed 3% PFA and 0.1% glutaraldehyde (GA, 1:80-diluted 8% glutaraldehyde solution, 16019, Electron Microscopy Sciences) in CBS at RT for 15 mins with gentle rocking, followed by rinsing twice with 1× PBS. Cell permeabilization was carried out using block buffer at RT for 30 mins with gentle rocking, after which the solution was aspirated. The cells were incubated with primary antibodies (1:500-diluted mouse anti-α-tubulin primary antibody (T5168, Sigma-Aldrich) in antibody dilution buffer) at 4° C. overnight and rinsed three times for 5 mins with wash buffer (0.05% Triton X-100 in 1× PBS). The cells were then incubated with secondary antibodies (1:500-diluted goat anti-mouse AF647 secondary antibody (ab150115, Abcam) in antibody dilution buffer) at RT for 30 mins and rinsed three times for 5 mins with wash buffer. Post-fixation was performed with the same buffer as fixation at RT for 10 mins with gentle rocking, followed by three rinses with 1×PBS. The cells were stored in 1×PBS at 4° C. until imaging. For immunofluorescence-labeled Rtn4, cells were fixed with pre-warmed 3% PFA and 0.5% GA in 1×PBS at RT for 15 mins with gentle rocking and rinsed twice with 1×PBS. Autofluorescence quenching was performed with fresh 0.1% sodium borohydride in 1× PBS at RT for 7 minutes with gentle rocking, followed by three rinses with 1×PBS. Cell permeabilization was carried out using 5% donkey serum (017000121, Jackson Immunoresearch) and 0.2% Triton X-100 in 1×PBS at RT for 30 mins with gentle rocking, after which the solution was aspirated. The cells were incubated with primary antibodies (1:200-diluted sheep anti-Rtn4b primary antibody (AF6034, R&D systems) in 5% donkey serum) at 4° C. overnight and rinsed three times for 5 mins with wash buffer. The cells were then incubated with secondary antibodies (1:400-diluted donkey anti-sheep AF647 secondary antibody (A21448, ThermoFisher Scientific) in block buffer) at RT for 10 mins and rinsed three times for 5 mins with wash buffer. Post-fixation was performed with the same buffer as fixation at RT for 10 mins with gentle rocking, followed by three rinses with 1×PBS. The cells were stored in 1×PBS at 4° C. until imaging.

Sample mounting of cells and preparation of imaging buffer

Right before imaging acquisition, 1 μL of 1:106-diluted 200-nm crimson beads (200 nm crimson beads, Invitrogen) in double-distilled water were added around the center region of the coverslips with labeled cells on top. The samples were mounted with 70 μL of a freshly prepared imaging buffer consisting of 10 mM 2-mercaptoethylamine (MEA, M9768, Sigma-Aldrich), 50 mM 2-hydroxyethylmercaptan (BME, 63689, Sigma-Aldrich), 2 mM cyclooctatetraene (COT, 138924, Sigma-Aldrich), 2.5 mM PCA, and 50 nM PCD in dSTORM buffer. Another coverslip was placed on top to form a sandwiched sample. The samples were then sealed using silicone dental glue and left to dry at RT for 30 mins prior to imaging.

For the preparation of cell-on-top-coverslip samples, cleaned coverslips were initially immersed in poly-L-lysine solution at RT for 30 mins and subsequently rinsed once with double-distilled water. These coverslips were then incubated with 100 μL of 1:106-diluted 200-nm crimson beads in double-distilled water at RT for 30 mins and rinsed again with double-distilled water, serving as the bottom coverslips in the sample sandwiches. For the top coverslips in the sample sandwiches, coverslips with labeled cells on top were treated with 1 μL of 1:106-diluted 200-nm crimson beads in double-distilled water around the center region. The treated top and bottom coverslips were then assembled with 70 μL of freshly prepared imaging buffer, sealed using silicone dental glue, and left to dry at RT for 30 mins prior to imaging.

### Immunofluorescence Labeling of Brain Sections

The 3.5-month-old transgenic mice (B6.Cg-Tg(Thy1-COP4/EYFP)18Gfng/J, The Jackson Laboratory) were anesthetized with an intraperitoneal injection of a mixture containing 90 mg/kg ketamine (59399-114-10, Akron) and 10 mg/kg xylazine (343750, HVS). The mice were transcardially perfused with 1×PBS, followed by 4% PFA to pre-fix the brains. The brains were carefully excavated from the skulls and immersed in 4% PFA overnight at 4° C. for further fixation. The following day, fixed brain samples were cut into 50-μm coronal sections using a vibratome (1000 Plus, Vibratome). The sections were stored at 4° C. in 1×PBS until labeling. For immunofluorescence-labeled Thy 1+pyramidal cells expressing ChR2-EYFP in mouse brains, the sections were rinsed with 0.1% Triton X-100 in 1×PBS three times for 15 mins with gentle rocking. They were then blocked with blocking buffer (5% BSA in 1× PBS) for 1.5 hours with gentle rocking. The sections were incubated with 1:1,000-diluted chicken anti-GFP antibody (ab13970, Abcam) in blocking buffer at 4° C. overnight and then rinsed three times for 15 mins with wash buffer (0.1% Triton X-100 in 1× PBS). Subsequently, the sections were incubated with 1:600-diluted goat anti-chicken AF647 secondary antibody (A21449, Invitrogen) in wash buffer at RT for 2 hours with gentle rocking. The sections were rinsed three times for 15 mins with wash buffer and stored in 1×PBS at 4° C. until imaging.

### Tissue Clearing for Single-Molecule Localization Microscopy

Tissue clearing is critical for high-resolution 4Pi-SMSN imaging, where light scattering and absorption degrade interferometric contrast, fluorescent signal strength, and background suppression. While tissue imaging protocols can largely follow those established for cultured cells once sufficient optical transparency is achieved, the inherent optical inhomogeneity of tissues limits the retrievable information from tissue imaging. To address this, tissue clearing methods have been developed to enhance transparency through lipid removal, decolorization, and decalcification, such as SeeDB, Scale, and iDISCO75-80. Established methods have demonstrated their ability to render brain tissue transparent while preserving structural integrity, enabling imaging depths from <20 μm to >150 μm with minimal sample distortion. However, not all methods are compatible with 4Pi-SMSN. Certain reagents commonly used in clearing protocols can disrupt the thiol-based imaging buffer required for single-molecule localization. In our experiment, tissue clearing methods incorporating sodium dodecyl sulfate, acrylamide hydrogel, dichloromethane, and dibenzyl ether affected fluorophore stability and single-molecule blinking, making them unsuitable for 4Pi-SMLM. In contrast, the fast optical clearing method (FOCM) 45 provided tissue transparency while maintaining acceptable blinking performance when integrated with thiol-based imaging buffers. The tissue clearing was performed by incubating brain section with 200 μL FOCM reagent (30% wt/vol urea (U15-500, Fisher Scientific), 20% wt/vol D-sorbitol (S1876-500G, Sigma-Aldrich), and 5% wt/vol glycerol (G5516, Sigma-Aldrich) in DMSO (276855, Sigma-Aldrich)) at RT for 1-2 mins.

### Sample Mounting of Brain Sections and Tissue Clearing

Right before imaging acquisition, the labeled brain sections were placed on cleaned coverslips, with the visual cortex regions positioned at the center of the coverslips. Tissue clearing was then performed and rinsed once with 1×PBS. 1 μL of 1:106-diluted 200-nm crimson beads in double-distilled water was added around the outer edge of the visual cortex. The samples were then assembled with 70 μL of freshly prepared imaging buffer, sealed using silicone dental glue, and left to dry at RT for 30 mins prior to imaging.

For the preparation of bead with unlabeled brain section samples, cleaned coverslips were initially immersed in poly-L-lysine solution at RT for 30 mins and subsequently rinsed once with double-distilled water. These coverslips were then incubated with 100 μL of 1:106-diluted 200-nm crimson beads in double-distilled water at RT for 30 mins and rinsed again with double-distilled water. The unlabeled brain sections were placed on the center of the coverslips, serving as the bottom coverslips in the sample sandwiches. For the top coverslips in the sample sandwiches, coverslips with labeled cells on top were treated with 1 μL of 1:106-diluted 200-nm crimson beads in double-distilled water around the center region. The treated top and bottom coverslips were then assembled with 70 μL of 1×PBS, sealed using silicone dental glue, and left to dry at RT for 30 mins prior to imaging.

### Imaging Acquisition of Fluorescent Beads and System Calibration

The bead sample was illuminated with a modest laser power of 30 W/cm² for calibration and imaging acquisition. The alignment process began with a preliminary alignment of the upper and lower objectives to coincide with fluorescent patterns acquired from them. Next, the upper and lower DMs were meticulously calibrated to introduce vertical astigmatism with amplitudes of #1λ/2π, preventing axial ambiguity, and minimizing systematic aberrations. To estimate the aberrations, beads were scanned within an axial range of +1 μm, and acquired using upper and lower objectives, respectively. The step size for this scan was 100 nm, and each position was recorded for 5 frames with an acquisition time of 100 ms. The resulting bead measurements were subjected to a phase retrieval algorithm to calculate the respective pupil functions and their corresponding Zernike decomposition81,82. To compensate for systematic aberrations, voltage maps corresponding to the opposite Zernike modes (Wyant order, up to 2nd-order aberrations) were applied to the DMs for calibration. To optimize the interferometric contrast in the coherent fluorescent detection, the tower mechanical system was scanned to measure the interferometric intensity profile at the bead's center under different cavity phases. The tower mechanical system was then positioned at the position where the maximal intensity modulation was observed. In the experiment assessing the practical performance of the in-situ 4Pi-PSF modeling, an interferometric bead measurement was conducted. The beads were scanned within an axial range of #1 μm. The step size was set at 100 nm, and each position was recorded for 50 frames, with an acquisition time of 20 ms. In addition to the non-aberrated measurements, 7 Zernike modes (Wyant order, ranging from vertical astigmatism to vertical trefoil) were introduced to the upper and lower DMs with amplitudes of +1λ/2π, creating aberrated interferometric patterns.

Imaging acquisition of biological samples and measurement of sample thickness The biological sample was illuminated with a modest laser power of 30 W/cm² for searching region of interest, after which the system was calibrated employing beads on the coverslip. For the construction of the in-vitro cubic-spine model, beads were scanned within an axial range of #1 μm to capture the interferometric patterns37. The step size for this scan was set at 20 nm, and each position was recorded for 5 frames with an acquisition time of 100 ms. In constructing the in-vitro phase-retrieval model38, individual pupil functions of both objectives were collected, using the same procedure in fluorescent bead imaging acquisition (Method 1.10), besides interferometric bead measurement.

Afterward, the samples were positioned to center the targeted structures, followed by exposure to a laser with a power of 12 kW/cm² for 1 min, transferring fluorophores into dark states and bleached autofluorescence. Subsequently, the laser power was reduced to 7 kW/cm² for imaging acquisition. For each FOV, 80,000-100,000 frames were collected with an acquisition time of 20 ms. Each frame was then cropped to yield four-channel detections. In the experiment evaluating the dynamic update capability of 4Pi in-situ PSF modeling, we collected 20 sequential datasets, each comprising 2,000 frames, following the above configuration. Prior to each collection window, random aberrations were introduced to the deformable mirrors in the upper and lower arms. These aberrations were generated across 10 Zernike modes (Wyant order, ranging from vertical tilt to vertical trefoil), with random amplitudes between #1λ/2π. To measure the sample thickness at the targeted position, the sample was incrementally moved through the focal plane. The distance between the bottom and top coverslip surfaces, identified by the presence of random dust particles on the top surface, was recorded as the sample thickness.

### Example 2: Systematic Configuration and Simulated Method

### 4Pi Single Molecule Localization Microscopy

The 4Pi single molecule imaging system was assembled based on a previously published schematic design, incorporating two 100×/1.4-NA oil-immersion objectives (UPLSAPO 100XO, Olympus) for coherent interferometric single-molecule detection. The emission path was configured to collect emissions from both objectives, which were divided into P- and S-polarized orientations with equal amplitudes using quarter-wave plates (AQWP05M-600-UM-SP, Thorlabs). The emissions were aligned along four coinciding paths using a combination of nonpolarized and polarized beam splitters (NT47-009 and NT49-002, Edmund Optics), and directed onto distinct regions of an sCMOS camera (Orca-Flash4.0v2, Hamamatsu) for simultaneous 4-channel acquisition. A telecentric optical design was employed, resulting in a final magnification of 50× and an effective pixel size of 129 nm, allowing for 4Pi interferometric imaging across a 20-μm field of view (FOV).

To reduce background signals in fluorescent acquisition, quad-band dichroic and bandpass filters (FF01-446/523/600/677-25, Di01-R405/488/561/635-17.5×24, Semrock) were positioned between the quarter-wave plates and the nonpolarized beam splitter. This arrangement separated the emission from the excitation. Aberrations in two interferometric arms were individually controlled using two deformable mirrors (DM) (Multi-5.5, Boston Micromachines), equipped with 140 actuators, placed at the conjugate pupil planes. To control phase delay and dispersion across the four optical paths, two customized Babinet-Soleil compensators were placed between the DMs and the nonpolarized beam splitter. The thickness of the compensators was optimized to achieve phase delays of 0, π/2, π, and 3π/2 with minimized dispersions between the upper and lower arms for four channels.

In excitation, a 642-nm continuous-wave laser (2RU-VFL-P-2000-642-BIR, MPB Communications) was used, whose power was controlled by an acousto-optic tunable module (AOTFnC-400.650-TN, AA Opto-electronic). The laser light was guided through a polarization-maintaining single-mode fiber (PM-S405-XP, Thorlabs) and focused on the back focal plane of the lower objective for Köhler illumination, generating an illumination area of 27 μm. A mirror positioned at the conjugate imaging plane allowed for switching tilted angles of illumination between 0° (Epi mode) and 54° (HILO mode).

For alignment of the interferometric cavity, the upper and lower objectives were mounted on piezo nano-positioning motors (N-664.3A, P-612.2SL, PI) for axial and lateral adjustments, respectively. The sample holder was arranged on a combination of piezo positioning motors (P-541.XYZ, M-227.10, M-686.D64, PI) for nanoscale sample positioning within a microscale range. A tower mechanical system (LS-50, ASI) facilitated microscale vertical positioning of the objective and sample positioning modules. The nonpolarized beam splitter was mounted on a rotation goniometric motor (M-GON40-U, M-RS65, TRA12CC, Newport) for fine-tuning the spatial coincidence of upper and lower fluorescence. The microscope setup was synchronized using a custom Lab VIEW program.

### Aberration Control and Calibration of Deformable Mirrors

The DM calibration followed established methodologies, where DMs are controlled by an orthogonal set of 32 mirror deformation modes (DM modes, corresponding to Zernike modes up to 4th-order spherical aberration). To measure the response of DM modes, 100-nm fluorescent beads were scanned within an axial range of ±1 μm to capture the single-molecule patterns corresponding to DM modes with amplitudes of ±1λ/2π. The step size for this scan was 400 nm, and each position was recorded for 3 frames with an acquisition time of 100 ms. The resulting bead measurements were subjected to a phase retrieval algorithm to calculate the respective pupil functions for certain DM modes.

To solve mismatched orientations of the two DMs, we decomposed the DM modes of each DM into Zernike modes with the same orientation. The pupil function of the mth DM mode Φ_m was decomposed into a series of Zernike modes, excluding Piston, X-tilt, Y-tilt, and defocus aberration, which was expressed as

$${Φ}_{m} ( {k}_{x} , {k}_{y} ) ≅ {\sum }_{n = 5}^{64} {c}_{n} {Z}_{n} ( {k}_{x} , {k}_{y} ) \qquad ( 1 )$$

where cₙ and Zₙ(kₓ, k_{y}) are coefficient and aberration phase of the nth Zernike mode.
By solving this equation with least-square minimization, each Zernike mode can be expressed as a linear combination of DM modes. To mitigate the impact of residual aberrations, the difference between the retrieved amplitudes at amplitudes of ±λ/2π was calculated and divided by 2. To assess the performance of DM control, we retrieved pupil functions from fluorescent bead measurements by applying 17 Zernike aberrations (following Wyant order, from vertical astigmatism to oblique quadrafoil) with amplitudes of ±1λ/2π. The beads were scanned over an axial range of ±1 μm, with a step size of 100 nm, and each position was recorded for 5 frames with an acquisition time of 100 ms. The retrieved pupil functions showed an error of less than 0.4λ/2π from the applied Zernike modes.
Characterization of sCMOS Camera

The characterization of the sCMOS camera, including parameters such as offset, variance, and gain for each pixel, was determined using established methodologies 85. For pixel i, the relationship between input photon X_i and the readout value Y_i can be described as

$${Y}_{i} = {g}_{i} \cdot Poisson ( {X}_{i} ) + Gaussian ( {o}_{i} , {σ}_{i}^{2} ) \qquad ( 2 )$$

where gᵢ is the gain of the pixel i, oᵢ and

$${σ}_{i}^{2}$$

are Gaussian mean and variance of the pixel i. Poisson(x) represents Poisson random process with mean of x, and Gaussian(o,σ²) represents Gaussian random process with mean of o and variance of σ².
The Gaussian mean oᵢ and variance

$${σ}_{i}^{2}$$

can be estimated with illumination photon xᵢ=0 as

$${\hat{o}}_{ι} = {E}_{t ∈ T} [ {y}_{i}^{t} \vert {x}_{i}^{t} = 0 ] \qquad ( 3 )$$

$${\hat{σ}}_{ι}^{2} = {Var}_{t ∈ T} [ {y}_{i}^{t} \vert {x}_{i}^{t} = 0 ] \qquad ( 4 )$$

where

$${x}_{i}^{m} and {y}_{i}^{m}$$

are the input photon and readout value of pixel i at frame t, t E T encompasses all valid frames. The gain factor gᵢ can then be estimated under different illumination photon levels xᵢ=Sₙ by solving the least-square minimization problem

$${\hat{g}}_{ι} = arg min {‖ B - {g}_{l} \cdot A ‖}^{2} wher e A = [ \begin{matrix}{E}_{t ∈ T} ( {y}_{i}^{m} \vert {x}_{i}^{m} = {s}_{1} ) - {\hat{o}}_{ι} \\ {E}_{t ∈ T} ( {y}_{i}^{m} \vert {x}_{i}^{m} = {s}_{2} ) - {\hat{o}}_{ι} \\ ⋮ \\ {E}_{t ∈ T} ( {y}_{i}^{m} \vert {x}_{i}^{m} = {s}_{n} ) - {\hat{o}}_{ι}\end{matrix} ] , B = [ \begin{matrix}{Var}_{t ∈ T} ( {y}_{i}^{m} \vert {x}_{i}^{m} = {s}_{1} ) - {\hat{σ}}_{ι}^{2} \\ {Var}_{t ∈ T} ( {y}_{i}^{m} \vert {x}_{i}^{m} = {s}_{2} ) - {\hat{σ}}_{ι}^{2} \\ ⋮ \\ {Var}_{t ∈ T} ( {y}_{i}^{m} \vert {x}_{i}^{m} = {s}_{n} ) - {\hat{σ}}_{ι}^{2}\end{matrix} ] \qquad ( 5 )$$

The estimation of this overdetermined system can be derived by Moore-Penrose pseudo-inverse as

$${g}_{i} = {( A {A}^{T} )}^{- 1} AB \qquad ( 6 )$$

To assess the characterization experimentally dye-on-coverslip samples were imaged under illumination levels ranging from 0 to 3 kW/cm² for 5 trials. For each trial, 2,000 frames were

$${σ}_{i}^{2} ,$$

recorded with an acquisition time of 20 ms. The oᵢ, and gᵢ were then estimated using Eq. 3-6.

### Coherence Emission of 4Pi-SMSN

The 4Pi coherent emission can be described through the superposition of fluorescent wave fields collected from opposing objectives with the coincided focal plane, forming constructive or destructive interferometric patterns. The pupil functions of the system represent the amplitude of fluorescent wave fields at the conjugated pupil plane can be expressed as

$$h ( {k}_{x} , {k}_{y} ) = \{ \begin{matrix}a ( {k}_{x} , {k}_{y} ) \cdot {e}^{i φ ( {k}_{x} , {k}_{y} )} , & if {k}_{x}^{2} + {k}_{y}^{2} \leq {( \frac{NA}{λ} )}^{2} \\ 0 , & if {k}_{x}^{2} + {k}_{y}^{2} > {( \frac{NA}{λ} )}^{2}\end{matrix} \qquad ( 7 )$$

where a(kₓ, k_{y}) and q(kₓ, k_{y}) are the magnitude and phase of the electric field at the pupil plane, respectively, kₓ and k_{y} are wave vectors along X and Y directions, NA is the numerical aperture of the objectives and A is the fluorescent wavelength in air. The phase of pupil function φ(kₓ, k_{y}), which include sample- and system-induced interferometric aberrations, can be decomposed into a series of Zernike modes as

$$φ ( {k}_{x} , {k}_{y} ) = {\sum }_{n = 1}^{N} {c}_{n} {Z}_{n} ( {k}_{x} , {k}_{y} ) , \qquad ( 8 )$$

where Zₙ(kₓ, k_{y}) is the nth Zernike mode, cₙ is corresponding amplitude, and N is the total number of Zernike modes. The complexity of interferometric electric fields in 4Pi emission, resulting from superposition of coherent pupil functions from the upper and lower objectives, h_{U} and h_{L}, can be described by

$${h}_{s 1} ( {k}_{x} , {k}_{y} , z ) = {h}_{U} ( {k}_{x} , {k}_{y} ) D ( - z ) {e}^{i π} + {I}_{t} {h}_{L} ( {k}_{x} , {k}_{y} ) D ( z ) {e}^{i ( {φ}_{sp} + {φ}_{0} )} {h}_{s 2} ( {k}_{x} , {k}_{y} , z ) = {h}_{U} ( {k}_{x} , {k}_{y} ) D ( - z ) + {I}_{t} {h}_{L} ( {k}_{x} , {k}_{y} ) D ( z ) {e}^{i ( {φ}_{sp} + {φ}_{0} )} {h}_{p 1} ( {k}_{x} , {k}_{y} , z ) = {h}_{U} ( {k}_{x} , {k}_{y} ) D ( - z ) + {I}_{t} {h}_{L} ( {k}_{x} , {k}_{y} ) D ( z ) {e}^{i ( π + {φ}_{0} )} {h}_{p 2} ( {k}_{x} , {k}_{y} , z ) = {h}_{U} ( {k}_{x} , {k}_{y} ) D ( - z ) + {I}_{t} {h}_{L} ( {k}_{x} , {k}_{y} ) D ( z ) {e}^{i {φ}_{0}} \qquad ( 9 )$$

where D(z)=e^{i2πk}^{_{z}}^{(k}^{ₓ}^{,k}^{_{y}}^{)z} is defocusing term of wave fields,

$${k}_{z} ( {k}_{x} , {k}_{y} ) = \sqrt{{( \frac{n}{2} )}^{2} = {k}_{x}^{2} = {k}_{y}^{2}}$$

is wave vector along Z direction, n is the refractive index of the medium, φₛₚ is the phase difference between s- and p-polarized fluorescence, φ₀ is cavity phase between upper and lower paths when the emitter is at the coincided focal plane, and Iₜ is the transmission efficiency ratio between two optical paths.

### Interferometric Modeling of 4Pi-PSF

The 4Pi-PSF can be represented as Fourier transform of interferometric electric fields. According to scalar diffraction theory, the intensity Him and amplitude Hm of a coherent 4Pi-PSF for channel m∈(s1, s2, p1, p2) can be described as

$${μ}_{m} ( x , y , z ) = {\vert {H}_{m} ( x , y , z ) \vert}^{2} , m ∈ ( s 1 , s 2 , p 1 , p 2 ) ) \qquad ( 10 )$$

$${H}_{m} ( x , y , z ) = {A}_{m} ( x , y , z ) \cdot {e}^{i {Φ}_{m} ( x , y , z )} = ℱ [ {h}_{m} ( {k}_{x} , {k}_{y} , z ) ] \qquad ( 11 )$$

where Aₘ(x, y, z) and Φₘ(x, y, z) are magnitude and phase of Hₘ(x, y, z), [symbol] denotes the Fourier transform operator, and hₘ(kₓ, k_{y}, z) is the interferometric wavefields for channel m in Eq. 9. In practical implementations, the interference may exhibit partial coherence, leading to reduced fringe contrast due to any incoherent fluorescence. To account for this, the partially coherent 4Pi-PSF μ₀ is expressed as a linear combination of coherent intensity μ_{I} and incoherent intensity μ_{C}, which can be described as

$${μ}_{0 m} ( x , y , z ) = a {μ}_{Im} ( x , y , z ) + ( 1 - a ) {μ}_{C} ( x , y , z ) \qquad ( 12 )$$

$${μ}_{C} ( x , y , z ) = {\vert ℱ [ {h}_{U} ( {k}_{x} , {k}_{y} ) D ( - z ) ] \vert}^{2} + {\vert ℱ [ {I}_{t} {h}_{L} ( {k}_{x} , {k}_{y} ) D ( z ) ] \vert}^{2} \qquad ( 13 )$$

where a is wavelength-dependent coherence factor between 0 and 1.

### Simulation of 4Pi-PSF and Determination of Hyperparameters

The 4Pi-PSF simulation began by initiating pupil functions of h_{U} and h_{L}, which incorporated optical aberrations by setting Zernike modes. The partially coherent 4Pi-PSF μ₀ₘ for channel m was then generated based on Eq. 9-12 with hyperparameters of NA, λ, n, φₛₚ, Iₜ, and a. To account for noise processing, multiplication of μ₀ₘ and photon counts serve as the input to model the readout values of the sCMOS camera in Eq. 2.

To simulate practical imaging conditions for characterizing our algorithmic performance, we determined the initiating hyperparameters from real data. The emission wavelength λ was estimated from acquisition of crimson beads with an emission spectrum similar to AF647 and optimized by minimizing step size estimation errors. The refractive indices n of the imaging buffer and immersion oil were measured to be 1.352 and 1.516, respectively, using an Abbe refractometer (334610, Thermo Spectronic). The transmission efficiency It was measured by single-molecule photon counts collected from the upper and lower objectives.

To estimate the coherence factor a, we employed published configuration based on Gaussian-weighted moment operators. The process begins with aligning 4-channel interferometric patterns. Gaussian-weighted moment operators m (z) at z position are calculated by

$${m}_{m} ( z ) = \int \int {D}_{m} ( x , y , z ) \sqrt{{x}^{2} + {y}^{2}} {e}^{- \frac{{x}^{2} + {y}^{2}}{2 {σ}^{2}}} dxdy \qquad ( 14 )$$

where Dₘ(x, y, z) is the acquired photons for channel m, and σ is predetermined root-mean-square (RMS) Gaussian width of 77 nm. According to 4Pi emission, the phase delays between channels with the same polarization are x rad. This implies that locally maximal and minimal values of two moment operators with the same polarization will occur at the same axial positions, respectively, where the coherence factor a can be estimated by calculating the maximal fringe contrast of

$$a = \frac{{a}_{p} + {a}_{s}}{2} , \qquad ( 15 )$$

$$where {a}_{k} = max \frac{\vert {m}_{k 1} ( z ) - {m}_{k 2} ( z ) \vert}{{m}_{k 1} ( z ) + {m}_{k 2} ( z )} , k ∈ ( s , p )$$

To estimate the phase difference between s- and p-polarized fluorescence φ_sp, we further express the moment operators M(z) as linear combinations of coherent terms and incoherent terms as

$$\begin{matrix}{M}_{m ′} ( z ) = {A}_{s} ( z ) [ {d}_{m}^{′} \pm {b}_{m}^{′} cos ( {k}_{z} z + {φ}_{s} ) ] , {m}^{′} ∈ ( s 1 , s 2 ) \\ {M}_{m ″} ( z ) = {A}_{p} ( z ) [ {d}_{m}^{″} \pm {b}_{m}^{″} sin ( {k}_{z} z + {φ}_{p} ) ] , {m}^{″} ∈ ( p 1 , p 2 )\end{matrix} \qquad ( 16 )$$

where Aₛ(z)=\sqrt{mₛ₁(z)²+mₛ₂(z)²} and Aₚ(z)=\sqrt{mₚ₁(z)²+mₚ₂(z)²} are the acquired modulation envelopes of s- and p-polarization, dₘ, and bₘ, are the incoherent and coherent terms of the operator modulation, k_{z} is modulation frequency, and φₛ and φₚ is phase delay of s- and p-polarization. The phase difference φₛₚ=φₛ−φₛ was estimated from normalized operator modulation using the ‘fminsearch’ function in MATLAB by solving the minimization problem as

$$\hat{θ} = arg min {\sum }_{m} {( \frac{{M}_{m} ( θ \vert z )}{{A}_{k} ( z )} - \frac{{m}_{m} ( z )}{{A}_{k} ( z )} )}^{2} , θ ∈ ( {φ}_{s} , {φ}_{p} ) \qquad ( 17 )$$

which was ranging from −1.5 to −1.7 rad in our setup.

In conducting simulations of randomly aberrated 4Pi-PSF, we set the amplitude of vertical astigmatism to −1.5 and +1.5λ/2π for the upper and lower pupils as prior knowledge. The amplitudes of other Zernike modes, ranging from oblique astigmatism to tertiary spherical aberration, were randomly sampled within a range of ±1λ/2π for 30 trials. In each trial, we generated 2,000 interferometric patterns at random axial positions ranging from ±800 nm with random lateral offsets of ±250 nm from the center within a sub-region size of 40 pixels. The photon count for each emission pattern was set to 2,000 and the background count per pixel was set to 5.

In conducting simulations of cavity phase estimation, the above aberration configuration was used to generate 60 trials. In each trial, we generated 2,000 interferometric patterns at random axial positions ranging from ±500 nm and random cavity phase ranging from ±π rad with random lateral offsets of ±250 nm from the center within a sub-region size of 16 pixels. The photon count for each emission pattern was set to 2,000 and the background count per pixel was set to 5. To further validate the robustness, we introduced a deviation between the input phase and the ground truth in the construction of the 4Pi-BRAINSPOT model. Subsequently, we employed the model with these deviations to estimate the original data, setting the deviations at 0, π/4, π/2, and 3π/4 (30 trials for each deviation).

In conducting simulations of objective misalignment estimation, the same aberration configuration was used to generate 60 trials. In each trial, we generated 2,000 interferometric patterns at random axial positions ranging from ±500 nm and random objective misalignment ranging from ±160 nm laterally and ±50 nm axially (corresponding to contravariant tilt and defocus aberrations of ±1λ/2π) with random lateral offsets of ±250 nm from the center within a sub-region size of 16 pixels. The photon count for each emission pattern was set to 2,000 and the background count per pixel was set to 5.

In conducting simulations of 4Pi channel-specific localization, we set the amplitude of vertical astigmatism to −2 and +2λ/2π for the upper and lower pupils. We generated 1,000 interferometric emission patterns at 11 axial positions ranging from ±500 nm with a step size of 100 nm within a sub-region size of 16 pixels. The photon count for each emission pattern was set to 2,000 and the background count per pixel was set to 30. To simulate system imperfection, the simulations were sequentially rotated by 15 degrees using an affine transformation and incorporated Poisson noise processing as well as pixel-dependent Gaussian noise in sCMOS camera.

### Example 3: Configuration of 4Pi-BRAINSPOT Algorithm

### Workflow of 4Pi In-Situ PSF Retrieval

The 4Pi in-situ PSF retrieval algorithm in the 4Pi-BRAINSPOT approach, inspired by our previously published in-situ PSF retrieval method, captures interferometric distortions present in observed interferometric patterns. This algorithm constructs the 4Pi-PSF library by cropping individual interferometric emission patterns from acquired 4-channel single-molecule blinking images and assigns acquired patterns to their corresponding positions accurately in the absence of latent 3D positions. These are fed into coherent 4Pi phase retrieval algorithm, retrieving the coherent pupil functions (FIG. 9). Through subsequent iterations, the coherent pupil functions were refined until stable coherent pupil functions were achieved. For the dynamic model update, we estimated and accommodated time-dependent interferometric aberrations according to objective misalignment in XYZ direction and cavity phase variation for each time window (Method 3.10&11). These coherent pupil functions were then used to construct a dynamic in-situ 4Pi-PSF model representing interferometric aberration information.

Alignment of Interferometric Channels and Construction of 4Pi-PSF Library from Single-Molecule Datasets

To ensure consistency across the dataset in 4-channel interferometric pattern processing, the raw 4-channel blinking datasets underwent an initial alignment prior to cropping emission patterns. This alignment involves using the maximum-intensity projection of 2,000 frames for each channel to calculate translation, scale, shear, and rotation operations between channel p1 and the other channels. These operations are estimated using affine transformation calculation via the ‘imregtform’ function in MATLAB. The resulting affine matrices are applied to align the other channels to channel p1 using the ‘imwarp’ function in MATLAB.

In cropping single-molecule patterns, the aligned datasets are superposed to relate the total acquired photon number across the FOV under incoherent conditions. To ensure each cropped pattern corresponding to individual single molecules, we follow a published configuration to check that maximum intensities exceed an initial intensity threshold Iᵢₙᵢₜ, the center distance between any two nearby subregions is larger than a distance threshold dₜₕᵣₑₛₕ, and the remaining intensities are higher than a segmentation threshold I_{seg}. The coordinates of candidate single-molecule patterns are then used to crop interferometric emission patterns from the 4-channel dataset to construct the 4Pi-PSF library.

### Axial-Position Assignment and Analysis of Coherent Interferometric Patterns

The axial-position assignment began by generating reference 4Pi-PSF patterns at a series of Z positions. In the first iteration, the pupil functions of h_{U} and h_{L} are initiated with hyperparameters used in 4Pi-PSF simulation, including the aberration of vertical astigmatism set to ±1.5λ/2π for simulation and ±1.2λ/2π for experiment as prior knowledge. These were used to generate 4Pi-PSF patterns at various axial positions ranging from ±1 μm, with a step size of 50-100 nm. In subsequent iterations, the pupil functions h′_{U} and h′_{L} were updated based on the results from the previous iteration.

The interferometric patterns in 4Pi-PSF library were classified into distinct groups based on their similarity to the reference PSFs. The interferometric similarity score Sᵢⱼ was calculated between pair i of reference 4Pi-PSF patterns μᵢ and pair j of acquired interferometric patterns Di across different channels by normalized cross-correlation (NCC) calculation, given by

$${S}_{i j} = \frac{1}{4} {\sum }_{m} N C C ( {μ}_{i , m} , {D}_{j , m} ) , where \qquad ( 18 )$$

$$NCC ( A , B ) = \frac{1}{N} {\sum }_{n} \frac{1}{{σ}_{A} {σ}_{B}} {A}_{n} {B}_{n}$$

where NCC denotes the normalized cross-correlation operator, n is value for pixel index n, N is the total pixel number of n, GA and OB are standard deviations of A and B. The interferometric patterns were assigned to a group with the highest similarity score. To ensure a robust analysis in following steps, we excluded interferometric patterns with a similarity score below Sₘᵢₙ of 0.5-0.6 and groups with a pair number below N_{g} of 5-30. The interferometric patterns within each group were subjected to an alignment process and averaged to form a single 4Pi-PSF observation at a certain Z position, mitigating the noise effect. 2D cross-correlation analysis is operated to calculate the subpixel shift from the 4-channel reference 4Pi-PSF patterns, respectively, utilizing Fourier interpolation with a 10-fold up-sampling. Patterns of each channel are then aligned and normalized using z-score normalization before averaging operation.

### Principle of Coherent 4Pi Phase Retrieval

Coherent 4Pi phase retrieval algorithm was devised to retrieve hidden interferometric information from acquired intensity profiles of interferometric emission patterns. Our methodology employing Gerchberg-Saxton algorithm estimates coherent pupil functions of conjugate objectives from coherent interferometric patterns at a series of Z positions, following the relationships in Eq. 12&13 (FIG. 9). To extract coherent patterns from partially coherent patterns, we employed prior knowledge of the coherence factor a and the incoherent emission patterns, computed by either averaging the 4-channel patterns or simulating from initial pupil functions of upper and lower objectives.

The algorithm begins by initiating pupil functions of h_{U} and h_{L} with hyperparameters used in 4Pi-PSF simulation. The magnitude Aₘ and phase Φₘ of coherent 4Pi-PSF amplitude Hₘ are calculated using Eq. 9-11. The amplitude H′m are updated by substituting magnitude Aₘ with the acquired magnitudes of coherent interferometric patterns at corresponding z positions as

$${H}_{m}^{′} ( x , y , z ) = \sqrt{{M}_{m} ( x , y , z )} \cdot {e}^{i {Φ}_{m} ( x , y , z )} ) \qquad ( 19 )$$

where Mₘ(x, y, z) is the measured intensity profiles of coherent interferometric pattern at z position. The interferometric wavefields h′m are updated by inverse Fourier transform of H′ₘ as

$${h}_{m}^{′} ( {k}_{x} , {k}_{y} , z ) = {ℱ}^{- 1} [ {H}_{m}^{′} ( x , y , z ) ] ) \qquad ( 20 )$$

The coherent pupil functions h′_{U} and h′_{L} is then estimated from interferometric wavefields by solving the least-square minimization problem following the relationship in Eq. 9 as

$$\hat{X} = arg min {‖ Y - AX ‖}^{2} \qquad ( 21 )$$

$$where Y = [ \begin{matrix}{h}_{s 1}^{′} ( {z}_{1} ) \\ {h}_{s 2}^{′} ( {z}_{1} ) \\ {h}_{p 1}^{′} ( {z}_{1} ) \\ {h}_{p 2}^{′} ( {z}_{1} ) \\ {h}_{s 1}^{′} ( {z}_{2} ) \\ ⋮ \\ {h}_{p 2}^{′} ( {z}_{n} )\end{matrix} ] , A = [ \begin{matrix}{e}^{i π} D ( - {z}_{1} ) & {I}_{t} {e}^{i ( {φ}_{sp} + {φ}_{0} )} D ( {z}_{1} ) \\ D ( - {z}_{1} ) & {I}_{t} {e}^{i ( {φ}_{sp} + {φ}_{0} )} D ( {z}_{1} ) \\ D ( - {z}_{1} ) & {I}_{t} {e}^{i ( π + {φ}_{0} )} D ( {z}_{1} ) \\ D ( - {z}_{1} ) & {I}_{t} {e}^{i {φ}_{0}} D ( {z}_{1} ) \\ {e}^{i π} D ( - {z}_{2} ) & {I}_{t} {e}^{i ( {φ}_{sp} + {φ}_{0} )} D ( {z}_{2} ) \\ ⋮ & ⋮ \\ D ( - {z}_{n} ) & {I}_{t} {e}^{i {φ}_{0}} D ( {z}_{n} )\end{matrix} ] , and X = [ \begin{matrix}{h}_{U}^{′} \\ {h}_{L}^{′}\end{matrix} ]$$

The estimation of this overdetermined system can be derived by Moore-Penrose pseudo-inverse as

$$\hat{X} = {( A {A}^{T} )}^{- 1} AY = [ \begin{matrix}{h}_{U}^{′} \\ {h}_{L}^{′}\end{matrix} ] \qquad ( 22 )$$

The updated coherent pupil functions h′_{U} and h′_{L} are used for the next iteration. After 60 iterations, the refined coherent pupil functions are regarded as the retrieved coherent pupil functions of the acquired interferometric emission patterns.

### Objective-Misaligned Interferometric Aberration Estimation

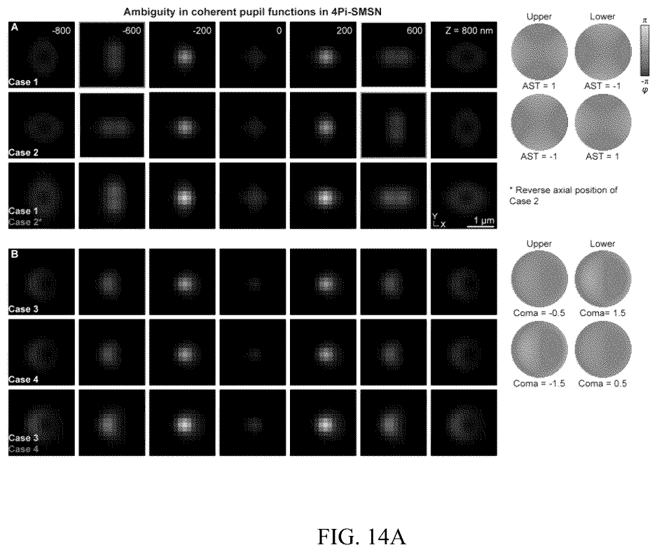

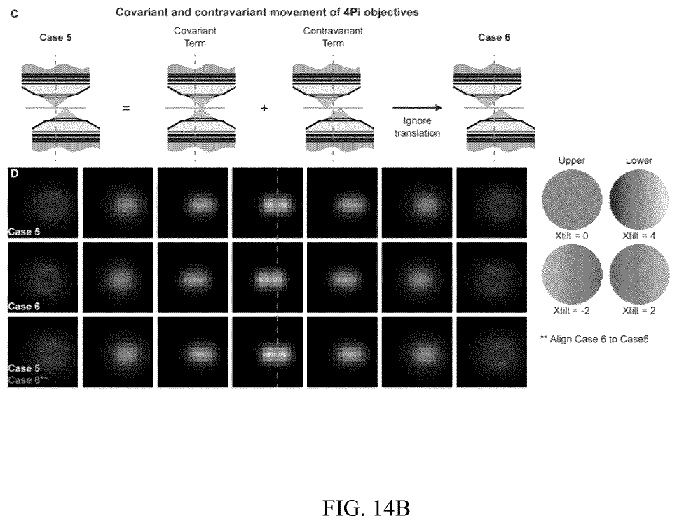

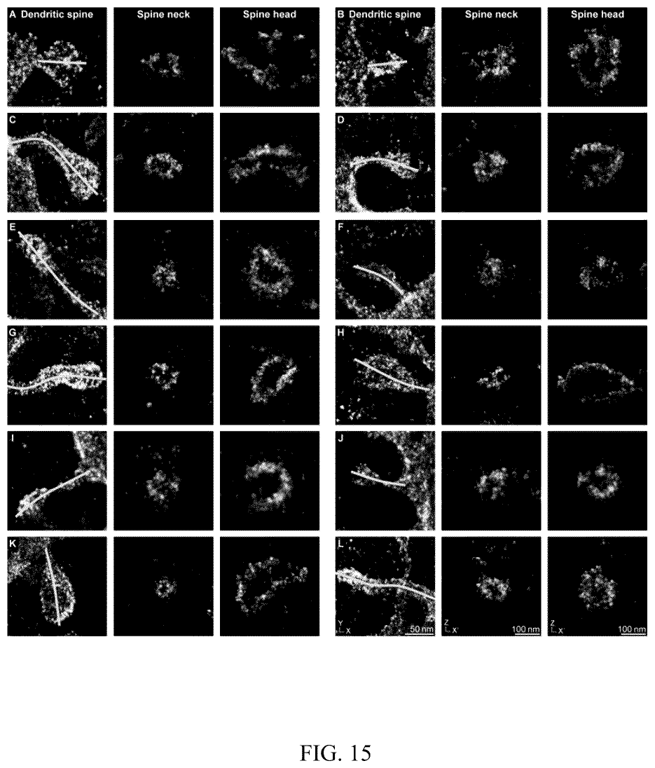

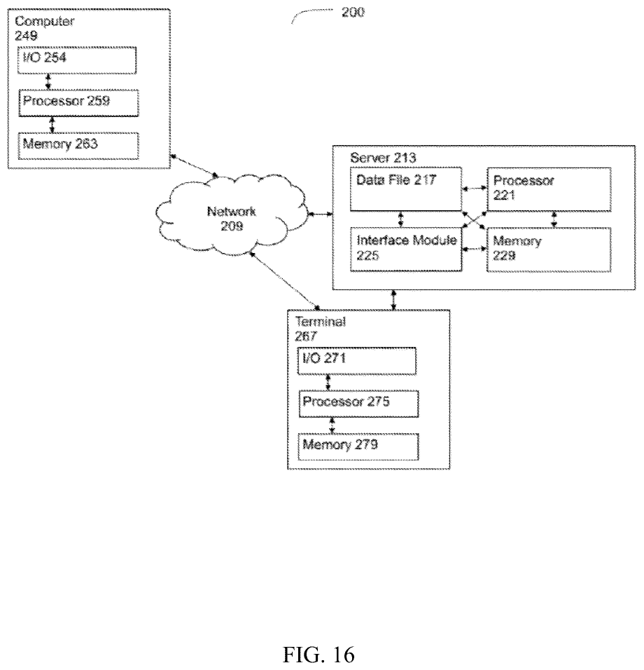

In the 4Pi microscopy system, objective drift in the XYZ directions results in interferometric aberrations, which can be categorized into covariant and contravariant terms. These terms are orthogonal to each other, that any drift in the 4Pi system can be represented by a unique combination of them (FIG. 14A panels A-B and FIG. 14B panels C-D). In our study, common lateral drifts and opposing axial drifts were defined as covariant terms, while the opposite configuration was defined as contravariant terms. The impact of covariant drift was manifested as a translation of the 4Pi-PSF without inducing interferometric aberrations, whereas contravariant drift modified the interferometric patterns, thereby causing time-dependent discrepancies in the 4Pi-PSF. To deal with these independent impacts, we implemented a dynamic 4Pi-PSF model update to address time-dependent changes in the diffraction patterns and utilize a drift correction algorithm to correct time-dependent translations.

To address contravariant objective misalignment in the in-situ 4Pi-PSF model, the 4Pi blinking dataset is segmented into several time windows, each with a duration of 40 seconds. We assumed that within each window, the interferometric patterns exhibit consistent aberrations. For each time window, a 4Pi-PSF library was constructed following Method 3.2. Subsequently, a series of reference 4Pi-PSF patterns are generated based on devised coherent pupil functions with additional contravariant misaligned aberrations ranging from ±1λ/2π. To circumvent the phase wrapping issue, these misaligned interferometric aberrations were calculated directly in the phase term and integrated into the coherence pupil functions. Each interferometric pattern pair was assessed the similarity between all reference 4Pi-PSF models using Eq. 18, and assigned with a misalignment coefficient corresponding to the model exhibiting the highest similarity. We determined the overall coefficients of respective time windows by applying Gaussian fitting to pinpoint the peak of the distribution. The procedure was iterated across all dimensions to refine the coherent pupil functions, reducing discrepancies between the captured patterns and the in-situ 4Pi-PSF model. To ensure a robust analysis, we excluded interferometric patterns with a similarity score below Sₘᵢₙ of 0.5-0.6 and axial positions exceeding ±500 nm.

### Estimation of Cavity-Phase-Induced Interferometric Aberration

The cavity phase characterizes the difference in optical path length from the common focal plane of the two objectives. To address time-dependent cavity phase variation, a dynamic in-situ 4Pi-PSF model update was incorporated across different time windows. We assumed that the interferometric patterns exhibit the same interferometric aberrations within each time window. For each time window, a 4Pi-PSF library was constructed. Afterward, a series of reference 4Pi-PSF patterns were generated based on devised coherent pupil functions with additional cavity phase variation ranging from ±π rad. Each interferometric pattern pair was assessed the similarity between all reference 4Pi-PSF models using Eq. 18, and assigned with a cavity phase corresponding to the model exhibiting the highest similarity. To enhance the accuracy of our analysis, particularly in addressing the phase wrapping issue observed around the boundary conditions of ±π rad, we employed periodic-padding Gaussian fitting. This method began with expanding the distribution of the cavity phase by shifting the phases ±2π rad from their original estimates, allowing a comprehensive representation of scores across cyclic cavity phases. We determined the overall coefficients of respective time windows by iteratively applying Gaussian fitting within a range of φᵢ₋₁±π rad, where φᵢ₋₁ was estimated cavity phase in the previous iteration, until stable value was achieved. For the first iteration, φ₀ was determined by mean of original estimates. To ensure a robust analysis, we excluded interferometric patterns with a similarity score below Sₘᵢₙ of 0.5-0.6 and axial positions exceeding ±500 nm.

### Principle of 3D Channel-Specific Single-Molecule Localization

To preserve the statistical properties of the raw camera characterization, we generated channel-specific 4Pi-PSF models for each detected channel to enhance single-molecule localization accuracy and minimize potential imaging artifacts. The process began with mapping coordinates in reference channel p1 into the other channels using calculated affine matrices in Method 2.10. For non-integer coordinates resulting from the affine transformation, the transformed coordinates were rounded down to the nearest integer. The round-off center coordinate (X′ₘ, Y′ₘ) for channel m, which was transformed from the center coordinate (Xₚ₁, Yₚ₁) of the reference channel p1 using the affine transformation matrix

$${[ \begin{matrix}a & b & 0 \\ c & d & 0 \\ e & f & 1\end{matrix} ]}_{m} ,$$

can be expressed as

$$( {X}_{m}^{′} , {Y}_{m}^{′} , 1 ) = ⌊ ( {X}_{m} , {Y}_{m} , 1 ) ⌋ = ⌊ {( {X}_{p 1} , {Y}_{p 1} , 1 ) [ \begin{matrix}a & b & 0 \\ c & d & 0 \\ e & f & 1\end{matrix} ]}_{m} ⌋ \qquad ( 23 )$$

$$\{ \begin{matrix}{X}_{m}^{′} = ⌊ {a}_{m} {X}_{p 1} + {c}_{m} {Y}_{p 1} + {e}_{m} ⌋ = {X}_{m} - Δ {X}_{m} \\ {Y}_{m}^{′} = ⌊ {b}_{m} {X}_{p 1} + {d}_{m} {Y}_{p 1} + {f}_{m} ⌋ = {Y}_{m} - Δ {Y}_{m}\end{matrix}$$

where └ ┘ denotes the floor operator, (Xₘ, Yₘ) is the exact transformed coordinate, subscript m is value for channel m, (a, b, c, d) represent the scaling, shearing, and rotation operations, (e, f) represents the translation operation, and ΔX and ΔY are round-off errors in X and Y direction. The coordinate relationship between the center of subregions of channel p1 & m can then be described as

$$( {X}_{m}^{′} + {x}_{m} , {Y}_{m}^{′} + {y}_{m} , 1 ) = {( {X}_{p 1} + {x}_{p 1} , {Y}_{p 1} + {y}_{p 1} , 1 ) [ \begin{matrix}a & b & 0 \\ c & d & 0 \\ e & f & 1\end{matrix} ]}_{m} \qquad ( 24 )$$

$$\{ \begin{matrix}{X}_{m}^{′} + {x}_{m} = {a}_{m} {X}_{p 1} + {c}_{m} {Y}_{p 1} + {e}_{m} + {a}_{m} {x}_{p 1} + {c}_{m} {y}_{p 1} \\ {Y}_{m}^{′} + {y}_{m} = {b}_{m} {X}_{p 1} + {d}_{m} {Y}_{p 1} + {f}_{m} + {b}_{m} {x}_{p 1} + {d}_{m} {y}_{p 1}\end{matrix}$$

where (xₘ>yₘ) are the position of single molecules in the cropped subregions of channel m. By substituting Eq. 23 into Eq. 24, the relationship between (xₚ₁, yₚ₁) and (xₘ>yₘ) can be expressed as

$$\begin{matrix}{x}_{m} = {a}_{m} {x}_{p 1} + {c}_{m} {y}_{p 1} + Δ {X}_{m} \\ {y}_{m} = {b}_{m} {x}_{p 1} + {d}_{m} {y}_{p 1} + Δ {Y}_{m}\end{matrix} \qquad ( 25 )$$

This analysis demonstrates that the cropped subregions, using round-off coordinates, share the same shape information, such as scale, shear, and rotation, as the original affine transformation. However, due to the rounding process, there may be translation discrepancies in the mapping from channel p1 to other channels. To compensate for these discrepancies, we applied a round-off calibration of ΔX and ΔY for each channel, ensuring correct characterization of the sCMOS camera was applied. Characterization maps of the camera were also cropped for each channel according to the round-off subregion coordinates.

After mapping the coordinates, we generated 4-channel in-situ 4Pi-PSF patterns at a center position of (xₚ₁, yₚ₁, z) using the coherent pupil functions retrieved from the in-situ 4Pi-PSF modeling process (Method 2.9). These patterns represent the 4Pi-PSF observations at the z position with channel-specific phase delays near 0, π/2, π, and 3π/2. We then 2D transformed these in-situ 4Pi-PSF patterns μₚ₁(xₚ₁, yₚ₁, z) into corresponding patterns μm (xₘ, yₘ, z) for channel m to ensure consistency across all channels. The transformation with affine matrix Tm calculated from Eq. 24&25 can be expressed as

$${μ}_{m} = {Warp}_{{T}_{m}} ( μ ) , {T}_{m} = {[ \begin{matrix}a & b & 0 \\ c & d & 0 \\ Δ X & Δ Y & 1\end{matrix} ]}_{m} \qquad ( 26 )$$

where Warp_{T}_{ₘ} denotes geometric transformation operator using the ‘imwarp’ function in MATLAB with affine matrix Tₘ. This transformation accounted for the calibrated translation discrepancies due to the rounding process. As a result, transformed patterns exhibited the channel-specific in-situ interferometric fringe at the exact corresponding coordinates. This ensured that the presence of Poisson noise and pixel-dependent readout noise in the sCMOS camera was accurately represented for each channel.

To conduct single-molecule localization, we assumed the photon numbers are evenly distributed across the four detected channels to reduce axial localization uncertainty. We utilized a maximum likelihood estimator (MLE) with a likelihood function L(θ|D) integrating channel-specific 4Pi-PSF models and accounting for the presence of Poisson noise and pixel-dependent readout noise in the sCMOS camera, which can be described as:

$$L ( θ \vert D ) = {\prod }_{m} {\prod }_{q} \frac{{( {μ}_{m , q}^{′} + {γ}_{m , q} )}^{( {D}_{m , q} + {γ}_{m , q} )} {e}^{- ( {μ}_{m , q}^{′} + {γ}_{m , q} )}}{Γ ( {D}_{m , q} + {γ}_{m , q} + 1 )} , θ ∈ ( x , y , z , I , bg ) \qquad ( 27 )$$

where θ is the parameter set including 3D single-molecule center position (x, y, z), photon number I, and background level bg, D is the acquired photon value of cropped interferometric pattern, μ′ is channel-specific 4Pi-PSF patterns,

$$γ = \frac{{σ}_{m , q}^{2}}{{g}_{m , q}^{2}}$$

is the noise character of the sCMOS camera, and subscript m, q is the value of pixel q for channel m. The parameters θ can be estimated using the modified Levenberg-Marquardt method by solving the minimization problem of negative log-likelihood function

$$\hat{θ} = argmin ℓ ( θ ) where ℓ ( θ ) = - ln ( L ( θ \vert D ) ) = {\sum }_{m} {\sum }_{q} {μ}_{m , q}^{′} - ( {D}_{m , q} + {γ}_{m , q} ) ln ( {μ}_{m , q}^{′} + {γ}_{m , q} ) + {C}_{m , q} \qquad ( 28 )$$

where C_{m,q} is constant of pixel q for channel m independent to θ. The estimation of this minimization problem can be iteratively estimated by

$$( 29 )$$

$${θ}_{n + 1} = {θ}_{n} - \frac{f}{{f}^{′} ( 1 + β )} \\ where \{ \begin{matrix}f = - \frac{\partial ln ( L ( θ \vert D ) )}{\partial θ} = {\sum }_{m} {\sum }_{q} ( 1 - \frac{{D}_{m , q} + {γ}_{m , q}}{{μ}_{m , q}^{′} + {γ}_{m , q}} ) \frac{\partial {μ}_{m , q}^{′}}{\partial θ} \\ f = \frac{\partial f}{\partial θ} = {\sum }_{m} {\sum }_{q} \frac{{D}_{m , q} + {γ}_{m , q}}{{( {μ}_{m , q}^{′} + {γ}_{m , q} )}^{2}} {( \frac{\partial {μ}_{m , q}^{′}}{\partial θ} )}^{2} = ( 1 - \frac{{D}_{m , q} + {γ}_{m , q}}{{μ}_{m , q}^{′} + {γ}_{m , q}} ) \frac{{\partial }^{2} {μ}_{m , q}^{′}}{\partial {θ}^{2}}\end{matrix}$$

ƒ and ƒ′ are 1^{st}- and 2^{nd}-order derivatives of negative log-likelihood function, and β is a damping factor related to convergence speed. Here, we set second derivative of

$$\frac{{\partial }^{2} {μ}_{m , q}^{′}}{\partial {θ}^{2}}$$

and β to be 0. To evaluate the data-model similarity, the log-likelihood ratio (LLR) was calculated between acquired interferometric emission patterns and corresponding channel-specific interferometric patterns in Eq. 27 as

$$\begin{matrix}LLR = - 2 ln ( \frac{L ( {μ}^{′} \vert D )}{L ( D \vert D )} ) \\ = 2 {\sum }_{m} {\sum }_{q} {μ}_{m , q}^{′} - {D}_{m , q} - ( {D}_{m , q} + {γ}_{m , q} ) ( ln ( {μ}_{m , q}^{′} + {γ}_{m , q} ) - \\ ln ( {D}_{m , q} + {γ}_{m , q} ) )\end{matrix} \qquad ( 30 )$$

### Precision Evaluation Using Cramer-Rao Lower Bound Calculation

To evaluate the quality of acquired interferometric pattern, we used published configuration⁸⁷ to calculate the Fisher information matrix F(θⱼ) for estimated parameters θⱼ under unbiased condition from the likelihood function in Eq. 27, which can be described as

$$F ( {θ}_{j} ) = {\sum }_{m} {\sum }_{q} \frac{1}{{μ}_{m , q}^{′} + {γ}_{m , q}} {( \frac{\partial {μ}_{m , q}^{′}}{\partial {θ}_{j}} )}^{2} \qquad ( 31 )$$

with Cramér-Rao lower bound (CRLB) precision σ_{θ} as

$${σ}_{θ}^{2} \geq \frac{1}{F ( {θ}_{j} )} \qquad ( 32 )$$

To calculate the Fisher information for channel-specific parameters, such as the single-molecule center position (xₘ, yₘ) across all channels, we used the calibrated transformation relationship from Eq. 26 when calculating the dependent derivatives in the Fisher information. For example, considering the Fisher information Fₘ(xₚ₁) for channel m, the 1^{st}-order derivative can be expressed as

$$\frac{\partial {μ}_{m , q}^{′}}{\partial {x}_{p 1}} = \frac{\partial {μ}_{m , q}^{′}}{\partial {x}_{m}} \frac{\partial {x}_{m}}{\partial {x}_{p 1}} + \frac{\partial {μ}_{m , q}^{′}}{\partial {y}_{m}} \frac{\partial {y}_{m}}{\partial {x}_{p 1}} = {a}_{m} \frac{\partial {μ}_{m , q}^{′}}{\partial {x}_{m}} + {b}_{m} \frac{\partial {μ}_{m , q}^{′}}{\partial {y}_{m}} \qquad ( 33 )$$

This leads to the following expression for the Fisher information for channel m as

$${F}_{m} ( {x}_{p 1} ) = {\sum }_{q} \frac{1}{{μ}_{m , q}^{′} + {γ}_{m , q}} {( {a}_{m} \frac{\partial {μ}_{m , q}^{′}}{\partial {x}_{m}} + {b}_{m} \frac{\partial {μ}_{m , q}^{′}}{\partial {y}_{m}} )}^{2} \qquad ( 34 )$$

Following the same process, the Fisher information Fₘ(yₚ₁) for channel m can be expressed as

$${F}_{m} ( {y}_{p 1} ) = {\sum }_{q} \frac{1}{{μ}_{m , q}^{′} + {γ}_{m , q}} {( {c}_{m} \frac{\partial {μ}_{m , q}^{′}}{\partial {x}_{m}} + {d}_{m} \frac{\partial {μ}_{m , q}^{′}}{\partial {y}_{m}} )}^{2} \qquad ( 35 )$$

### Addressing Ambiguities in 4Pi In-Situ PSF Retrieval

Ambiguity in 4Pi-SMSN appears when multiple sets of variables lead to the same observation of interferometric patterns. Here, we examined the ambiguity issues because of the symmetric nature of the 4Pi approach and how our methodology addressed the ambiguity issues to ensure clarity in analyzing the interferometric patterns.

The first ambiguity in 4Pi-SMSN arises from the axial indistinguishability when the 4Pi-PSF exhibits axial symmetry because of coupling between axial position and symmetric interferometric aberrations. For instance, the interferometric patterns of a single molecule at z=600 nm, with vertical astigmatisms of ±1λ/2π in the upper and lower pupil functions, appear identical to the patterns observed at z=−600 nm with vertical astigmatisms of ∓1λ/2π (FIG. 14A panel A). This ambiguity poses challenges to axial-position assignment process in the 4Pi phase retrieval algorithm, as interferometric patterns cannot be uniquely assigned to specific axial positions without additional information about coherent pupil functions or ground-truth single-molecule axial positions. To resolve this issue, we introduced vertical astigmatism with specific amplitudes as prior knowledge, breaking the indistinguishability between axial position and symmetric interferometric aberrations.

The second ambiguity arises from the distinguishability of coherent pupil functions, when interferometric aberrations contribute equally to both pupil functions. For instance, the interferometric patterns with vertical coma aberrations of −0.5 and 1.5λ/2π in the upper and lower pupil functions, respectively, appear identical to the patterns with coma aberrations of −1.5 and 0.5λ/2π (FIG. 14A panel B). This ambiguity poses challenges in the coherent pupil functions update process in the coherent 4Pi phase retrieval algorithm, as interferometric wavefields cannot be uniquely decomposed into specific coherent pupil functions without information about ground-truth pupil functions. Although this ambiguity cannot be broken up under 4Pi-BRAINSPOT framework, the distinguishability of coherent pupil functions does not influence the uniqueness in the representation of the 4Pi-PSFs. To prevent this ambiguity in the localization process and other analyses, we exclusively focused on interferometric patterns when interpolating the 4Pi-PSF model.

Another ambiguity arises from the distinguishability of coherent pupil functions, particularly when translating interferometric aberrations induced by common-term lateral objective drifts and opposite-term axial objective drifts. For instance, the interferometric patterns with horizontal tilt aberrations of 0 and 4λ/2π in the upper and lower pupil functions, respectively, appear identical to the patterns with horizontal tilt aberrations of −2 and 2λ/2π once we ignore the translation (FIG. 14B panel C-D). This ambiguity poses challenges in estimating objective-misaligned interferometric aberrations in the dynamic 4Pi-PSF model update process, as translating interferometric aberrations cannot be uniquely determined without information about ground-truth coherent pupil functions or ground-truth single-molecule positions. Although this ambiguity cannot be resolved within the 4Pi-BRAINSPOT framework, the translating interferometric aberrations don't influence the uniqueness in the representation of the 4Pi-PSFs. To prevent this ambiguity, we excluded translating interferometric aberrations in the dynamic 4Pi-PSF model update process and compensated for its influence through drift correction at the end of the localization process. After removing the translation ambiguity, we can utilize the unique amplitude of objective misalignment, including x tilt, y tilt and defocus, to characterize the overall movement of the objectives (FIG. 10A panels A-B and FIG. 10B panels C-D).

### Drift Correction in 4Pi-BRAINSPOT Reconstruction and Evaluation of Localization Reliability

To ensure the reliability of the reconstructed images from 4Pi single-molecule localization, we applied filtering criteria based on the CRLB, LLR, axial range of localization, and photon counts. We excluded localizations of interferometric patterns with a CRLB estimation above 10 nm for all three dimensions, an LLR below 2400, axial positions exceeding ±600 nm, and photon numbers below 1500 to ensure that only high-quality localizations contribute to the reconstructed image.

Drift correction was carried out using a redundant cross-correlation configuration²⁸. We divided the localizations into n segments, with each segment containing between 1,000 and 2,000 frames, and rendered them as n 3D volumes. 3D cross-correlation analysis was operated to calculate the subpixel shift between any two segments utilizing Fourier interpolation with a 10-fold up-sampling, resulting in total

$$\frac{1}{2} n ( n + 1 )$$

shift estimations. We determined the n−1 unknown 3D shift between adjacent segments from the overdetermined estimations and performed 3D drift correction to each segment. To further enhance the alignment accuracy in 3D reconstruction, additional drift correction was performed along the xz, yz, and xy directions, respectively.

### Imaging Rendering of 4Pi-BRAINSPOT Reconstruction

In imaging rendering of 2D images using MATLAB, the localizations were divided into 64 segments based on their axial positions. Depending on rendering transverse or vertical cross section, lateral or axial localization coordinates in each segment were mapped onto 2D images with a pixel size of 2-3 nm and pseudo-colored according to the axial position of the segment. In cases where multiple localizations were located at the same pixel, their values were accumulated. To blur the images, we applied a 2D Gaussian blur with 0.7-1 RMS width of overall CRLB precision. These blurred images were then merged to form a composite image. To enhance image contrast, pixel intensities were linearly scaled from the original range [0, L_{high}] to [0, 255], where L_{high} is a determined upper limit for imaging rendering. Lastly, we further stretched the image contrast to saturate the top 1-5% of pixels with the highest intensity values, minimizing contrast variation caused by exceptionally bright pixels.

In the 3D reconstruction process using Blender, we represented each localization as a particle positioned at its corresponding coordinates. These particles were pseudo-colored based on their axial positions to provide depth information. To visually represent the uncertainty associated with each localization, we rendered each particle as a two-layered sphere. The inner sphere had a diameter equal to the CRLB precision and was rendered with high opacity, while the outer sphere, with a diameter twice that of the inner sphere, was rendered with low opacity. To enhance the overall image contrast and reduce the impact of exceptionally bright pixels, we saturated the top 1-5% of pixels with the highest intensity values.

### Example 4: Quantification and Statistical Analysis

Analysis pipeline of 31) structural morphology and circumference

To accurately measure the morphology along interested structures from single-molecule localizations, we devised a semi-auto analysis pipeline for spine quantification. This process began by mapping the localization coordinates onto a 2D image with a pixel size of 10-20 nm, allowing users to identify the general spatial locations of the structures of interest by marking along them. The algorithm then traced these marks as a smooth curve using the ‘cubicspline’ fitting method in MATLAB and generated segments along the curves with dimensions of 200-600 nm in width, 600-1000 nm in height, and 150 nm in length. To prevent structural discontinuities due to the inherent sparsity of 4Pi-SMSN, we introduced a 50-nm overlap between adjacent segments.

Within each segment, the algorithm delineated the major structure using the ‘alphaShape’ algorithm in MATLAB, generating complex boundaries by uniting multiple convex hulls with an alpha radius of 0.3-0.5 in the cross-section encompassing subsets of localizations. Localizations falling within the structural region were fitted with a 2D elliptical function to locate the structural center. To minimize the impact of random outliers in radius measurement, we measured the distances between the structural center and all localizations within each segment, generating histograms with a bin size of 4-8 nm, and identified the mode of distances by fitting the histogram with a Gaussian function. The mode of distances was regarded as the geometric mean of radius. The volume trace was generated by fitted circular areas calculated from the geometric mean of radius using the ‘smoothingspline’ fitting method in MATLAB. To quantify the distribution of localizations on a structural surface, we sliced the straightened structures along their longitudinal direction and flattened them into a 2D surface. The localizations were then mapped to their corresponding angular positions, generating a 2D circumference plot color-coded according to angular positions ranging from ±π.

### Analysis of PSF Relevance and Data Visualization

To compare the similarity between two diffraction patterns, we calculated the similarity score S_{3D} between two interferometric patterns A and B in three dimensions by Eq. 18 as

$${S}_{3 D} = \frac{1}{4 Z} {\sum }_{z} {\sum }_{m} N C C ( {A}_{m , z} , {B}_{m , z} ) \qquad ( 36 )$$

where A_{m,z} and B_{m,z} are interferometric patterns at z position for channel m., and Z is total number of z. In the evaluation of localization uncertainty, we assessed the accuracy and precision of our system through fluorescent bead measurement. The accuracy and precision at each step were determined by calculating the mean absolute deviations and standard deviations, respectively, between the localized positions of the beads and their known ground truth positions.

In a box-chart visualization, the central line represented the median of the dataset and box represented upper and lower quartiles. The whiskers extended from the box to the furthest data points excluding outliers. Outliers were defined as values greater than q₃+1.5×(q₃−q₁) or less than q₃−1.5×(q₃−q₁). In a bar chart visualization, the bar heights represented the mean of the dataset, while the error bars indicated the standard deviation. For statistical significance, the p-values were calculated using ‘ttest2’ function in MATLAB, where the p-values smaller than 0.001 are denoted as p<0.001.

### Measurement of Illumination Profile in 4Pi-BRAINSPOT and Calculation of Signal-to-Background Ratio

The dye-on-coverslip sample was illuminated with a modest laser power of 30 W/cm² for imaging acquisition, after which system was calibrated employing single-molecule fluorophores on coverslips. To measure the tilted angle of illumination from the lower objective, the illumination area was reduced to a diameter of 7 μm, preventing clipping by FOV. The dye samples were scanned within an axial range of ±25 μm and acquired using upper objective with a synchronized scan. The step size for this scan was 2 μm, and each position was recorded for one frame with an acquisition time of 500 ms. For each measured image, illuminated regions were identified by calculating the median intensity as a threshold. We marked pixels with intensities above this threshold and generated patterns to encompass the marked pixels. The patterns were fitted with a 2D elliptical function to determine their centers and diameters at different axial positions. The tilted angle of the illumination profile was subsequently calculated based on the center orientation of the illumination within an axial range of ±15 μm. To estimate the Signal-to-Background Ratio (SBR), we simulated a 40×40×50 μm³ sample volume, with a unit cube size of 10 nm, containing uniformly distributed fluorophores. We assumed the imaging plane was located at the center of this volume and that fluorescence emission was linearly proportional to the illumination intensity. In the case of epi-illumination, we simulated uniform illumination throughout the entire volume. For HILO illumination, we modeled a light sheet with a Gaussian intensity profile perpendicular to its propagation direction, with a 1/e² width of 22.4 μm and intersecting the sample at an angle of 54° to the optical axis. During detection, the FOV was 20 μm, and fluorescence originating within an axial range of ±800 nm from the imaging plane was considered the signal. Fluorescence originating from regions outside this range was considered background. The detected emissions were calculated by aggregating contributions from different depths. For each depth, the fluorescent contribution was simulated by convolving the out-of-focus PSF pattern with the fluorescence map, which was determined as the product of the fluorophore density and the illumination profile. Consequently, the SBRs of 54°-HILO and 0°-Epi illumination were 8.0% and 3.2%, respectively, indicating a 2.5-fold improvement with a 60% reduction in background volume for a 50-μm tissue sample. Following the same simulation for an 80-μm-thick sample, it would be a 4.3-fold SBR improvement with a 77% background reduction.

Measurement of Intensity Profile from 4Pi-SMSN Dataset

To accurately measure the intensity profile of single-molecule localizations, we applied a histogram measurement to the localizations. This process began with mapping the localization coordinates onto a 2D image with a pixel size of 1.3 nm, enabling users to manually identify the structures of interest. The algorithm generated box segments along the line with a size of 4-6 nm and accumulated the number of localizations inside each segment, creating histogram profiles along the structures. To quantify the features, we fitted n Gaussian functions to n local maximum in the histogram, whose centers and FWHMs were regarded as the center positions and widths of features. To ensure robust analysis, we excluded features with localization counts below 2 and widths below estimated resolution. This approach obtained a nanoscale measurement of the intensity profile by minimizing imaging artifacts caused by the blurred kernel in the rendering process. To compare the intensity profiles of the same structures using various localization methods, we initially aligned the reconstructions using a redundant cross-correlation configuration and drew lines across similar structures, minimizing differences in the profiles due to global shifts between the reconstructions.

### Quantification of Cluster of Single-Molecule Localization

To identify single-molecule clusters, we assumed that localizations originating from a single molecule follow the 3D Gaussian random process with standard deviations of the overall CRLB precisions. The single-molecule clusters were determined once subsets of localizations fell within three standard deviations with no localization between three and five standard deviations. Statistically, when there are 5 observations in a cluster, the hit probability that all localizations will fall within three standard deviations is 98.7%. Conversely, the miss probability that at least one localization will fall between three and five standard deviations is 1.3%. To evaluate the size of clusters, the identified clusters were aligned using their center positions and superposed as a single cluster. The size of the superposition was determined by fitting with 2D Gaussian functions to calculate FWHM. To validate the accuracy of cluster size measurements, we also evaluated mean and standard deviation across all cluster sizes. To ensure robust analysis, we excluded clusters with a localization number below 4 and a total photon number below 8000.

### Quantification of 4Pi-SMSN Resolution

In this work, three different resolution quantifications were used. In the directional Fourier correlation (DFC) analysis, we segmented the localizations into 11×11 grids based on their lateral coordinates. Within each segment, the localizations were randomly divided into two statistically independent subsegments. The localization coordinates of each subsegment were mapped onto 3D images with a volume size of 3 nm. To prevent boundary issues, a 3D Tukey window function was applied to each 3D image. For each segment, the spatial frequency components along the certain direction of the two subsegments were extracted for correlation calculation. For a spatial frequency vector q [symbol] the directional Fourier correlation was defined as

$$DFC ( \vec{q} ) = \frac{{Σ}_{i ∈ I} \vert ( \vec{q} ) {( \vec{q} )}^{*}}{\sqrt{{Σ}_{i ∈ I} \vert ( \vec{q} ) {\vert}^{2} {Σ}_{i ∈ I} \vert ( \vec{q} ) {\vert}^{2}}} \qquad ( 37 )$$

where [symbol] ( [symbol] ) and [symbol] ( [symbol] ) were the spatial frequency components at frequency [symbol] in subsegments of segment i and i∈I encompasses all valid segments. The resulting DFC curves were then smoothed using the ‘cubicspline’ fitting method in MATLAB to reduce noise and facilitate accurate resolution determination. The DFC resolution for each segment was determined as the inverse of the spatial frequency at which the smoothed DFC curve reached the 1/7 criteria. To ensure robust analysis, segments were excluded with localization numbers below 200.

The Fourier shell correlation (FSC) and imaging decorrelation analysis were performed based on published configurations. In the FSC analysis, we segmented the localizations into 11×11 grids based on their lateral coordinates. Within each segment, the localizations were randomly divided into two statistically independent subsegments. The localization coordinates of each subsegment were mapped onto 3D images with a volume size of 3 nm. To prevent boundary issues, a 3D Tukey window function was applied to each 3D image. The FSC calculation for each segment was performed between the 3D Fourier transforms of the two subsegment images. This process involves computing the cross-correlation of corresponding shells in Fourier space and normalizing it by auto-correlation of each shell. The resulting FSC curves were then smoothed using the ‘cubicspline’ fitting method in MATLAB to reduce noise and facilitate accurate resolution determination. The FSC resolution for each segment was determined as the inverse of the spatial frequency at which the smoothed FSC curve reached the 1/7 criteria. To ensure robust analysis, segments were excluded with localization numbers below 200.

In the imaging decorrelation analysis, we also segmented the localizations into 11×11 grids based on their lateral coordinates. Within each segment, the localization coordinates were mapped onto monotonic 2D images with a pixel size of 3 nm and blurred using a 2D Gaussian blur with RMS width of overall CRLB precision. The decorrelation analysis for each segment involved computing the cross-correlation between the Fourier transform of the image and its binary circular mask-filtered version, under a series of Fourier low-pass and high-pass filtering. The imaging decorrelation resolution for each segment was determined by

$$\frac{2 \times Pixel size}{cuf - off frequency} .$$

This cut-off frequency identifies the highest spatial frequency at which a local maximum occurs in the decorrelation curves, indicating the spatial frequency beyond which the signal is no longer preserved. To ensure robust analysis, segments were excluded with localization numbers below 200.

### Example 5: Additional Embodiments

### Performance of In-Situ PSF Retrieval

FIG. 7 panels A-G show interferometric pattern comparison between fluorescent bead measurements (top row) and 4Pi-BRAINSPOT PSF models (bottom row) in channel pl with different aberrations at axial positions ranging from ±1 μm. Retrieved coherent pupil functions are shown on the right. FIG. 7 panel H shows a heatmap of NCC similarity between measured and modeled 4Pi-PSFs in (Panels A-G). FIG. 7 panel I shows stepwise localizations of bead measurements with 100 nm increments and 50 measurements per step over an axial range from ±1 μm using 4Pi-BRAINSPOT. Orange dot is localization result of 4Pi-BRAINSPOT, and gray dot is assigned stage position. Inset shows enlarged result in cyan box. FIG. 7 panel J shows Estimation of bias (top row) by calculating mean distances of localization results from assigned stage positions and precision (bottom row) by calculating standard deviations of localization results in (I). Orange curve is localization result, and gray dashed line is the stage accuracy.

Comparative Analysis of 4Pi-BRAINSPOT in Mouse Brain Slices with and without Tissue Clearing Process

FIG. 8 panels A-B show reconstruction and cross-sections of selected positions (yellow dashed line) of AF647-labeled ChR2 in PBS-immersed (Panel A) and tissue-cleared (Panel B) 50-μm thick mouse brain slices using 4Pi-BRAINSPOT, color-coded according to the single-molecule Z positions. Signal-to-background ratio (SBR) of tissue-cleared specimens was 106±15 (n=12 datasets) and that of untreated specimens was 80±6 (n=6 datasets), respectively. FIG. 8 panels C—H show enlarged views of selected regions (white dashed boxes) in (A&B). The lateral and axial localization of tissue-cleared specimens are 6.4±0.4 and 2.9±0.4 (n=12 datasets), while those of untreated specimens are 8.3±0.1 and 5.4±0.2 (n=6 datasets).

### Workflow of Coherent 4Pi Phase Retrieval Process

FIG. 9 shows coherent 4Pi phase retrieval began with initial coherent pupil functions with predetermined parameters. Superposed wavefields were generated by adding variable defocuses and phase delays across channels at axial positions ranging from ±800 nm. Amplitudes of 4Pi-PSF were computed by Fourier transform of superposed wavefield. Next, calculated magnitudes were replaced with the square root of obtained values, while maintaining original phases. Inverse Fourier transform was used to update superposed wavefields after which coherent pupil functions were updated by Moore-Penrose pseudo-inverse process. Results after 60 iterations were regarded as coherent pupil functions of obtained interferometric patterns.

### Dynamic Model Update and Channel-Specific Localization

FIG. 10A panel A shows illustration of dynamic model update in each time window. 4Pi-BRAINSPOT estimated and accommodated time-dependent interferometric aberrations according to objective misalignments (ΔX, ΔY, and ΔZ) and cavity phase shift (φ0). FIG. 10A panel B shows data processing workflow of dynamic in-situ model update in 4Pi-BRAINSPOT. The algorithm segmented individual 4Pi emission patterns from single-molecule datasets to create a library. We generated a series of reference PSFs for each time window based on coherent pupil functions from the previous window, with adjustments of 3D objective misalignments (ΔX, ΔY, and ΔZ) and cavity phase shifts (φ0). For each reference, NCC similarity score between obtained patterns and reference 4Pi-PSFs are evaluated to determine the time-dependent parameters. For objective misalignments, the corresponding coefficients are determined by fitting the similarity scores with a Gaussian function, identifying the peak position. For cavity phase variation, the corresponding coefficients are determined by periodic-padding Gaussian fitting the similarity scores and identifying the peak position. Refined results are regarded as retrieved coherent pupil functions for a given time window. FIG. 10B panel C shows data processing workflow of 3D channel-specific single-molecule localization. The process began with mapping coordinates in reference channel p1 into the other channels using calculated affine matrices. Channel-specific 4Pi-PSF models representing respective statistical properties of SCOMS are generated based on dynamic in-situ 4Pi-PSF model and used in further 4Pi localization. FIG. 10B panel D shows comparison of localization bias and precision between 4Pi-BRAINSPOT using channel-specific 4Pi-PSF models (orange) and translating acquired patterns (blue) instead. Insets are the simulated variance distribution.

### Quantitative Analysis of Single-Molecule Localization Clusters

FIG. 11 panel A shows reconstruction of AF647-labeled TOM20 in COS-7 cells in FIG. 3Q, color-coded according to the single-molecule Z positions. FIG. 11 panel B shows illustration of single-molecule cluster identification criteria. Magenta ellipse is region within three times of localization precision, yellow ellipse is region between three and five times of localization precision. Clusters were considered to originate from single molecules if more than three localizations fall in magenta region and none fall in yellow region. Red dot is simulated 7 localizations originated from single molecule and grayscale distribution is rendered cluster using 2D Gaussian blur. FIG. 11 panel C shows cross-sectional views cluster superposition (n=3244 localizations). Red dots are single-molecule localizations, grayscale distributions are rendered images of localization cluster, and blue lines are FWHMs of localization distributions. Horizontal (top) and vertical (bottom) cross-sections of superposed single-molecule cluster. Red dot is single-molecule localization (n=3244 localizations) and grayscale distribution is rendered cluster using 2D Gaussian blur with estimated FWHM. FIG. 11 panel D shows horizontal (top) and vertical (bottom) normalized intensity profiles of superposed single-molecule cluster. Red dot is single-molecule localization (n=3244 localizations), orange curve is fitted Gaussian function, and blue line marks estimated FWHM. FIG. 11 panel E shows lateral and axial FWHMs of all single-molecule clusters (10.3±3.0 and 5.3±1.9 nm, n=387 clusters). Gray error bar indicates mean and standard deviation of lateral and axial FWHMs. FIG. 11 panel F shows histograms of localization counts per cluster (top) and photon counts per cluster (bottom). FIG. 11 panels G-J show cross-sections and normalized intensity profiles for four selected clusters in (E) with >7 localizations each. Red dot is single-molecule localization (n=1845 localizations), grayscale distribution is rendered cluster using 2D Gaussian blur with estimated FWHM, orange curve is fitted Gaussian function, and blue line marks estimated FWHM.

### Resolution Assessment of 4Pi-BRAINSPOT in Mammalian Cells

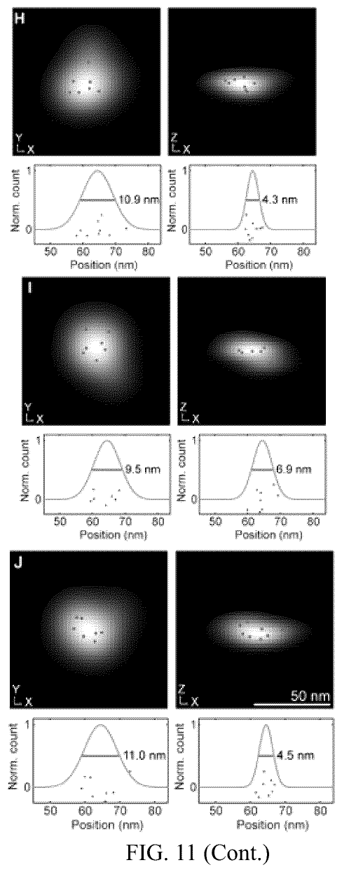

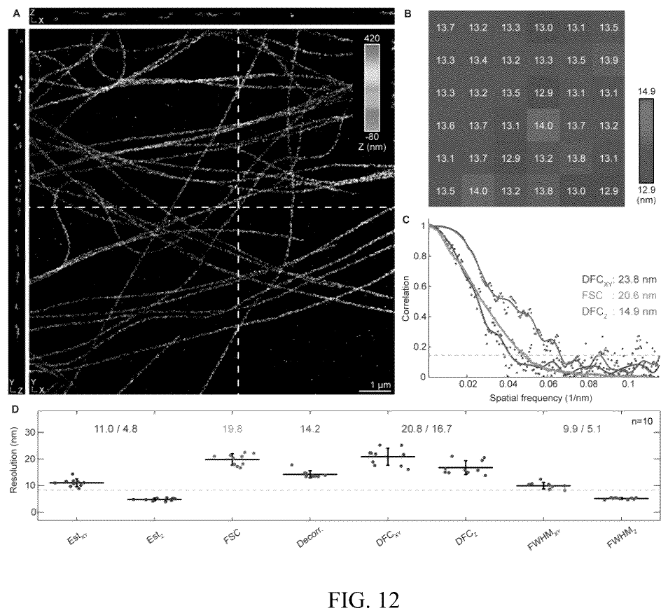

FIG. 12 panel A shows reconstruction and cross-sections of selected positions (white dashed lines) of AF647-labeled α-tubulin in COS-7 cells with a 25-μm cavity using 4Pi-BRAINSPOT, color-coded according to the single-molecule Z positions. FIG. 12 panel B shows heatmap of image decorrelation resolutions by segmenting 2D projection of (Panel A) into 6×6 subregions. FIG. 12 panel C shows correlation analysis of Fourier shell correlation (FSC, orange) and directional Fourier correlation laterally and axially (DFCXY, purple; DFCZ, navy) at different spatial frequencies. Marker is correlation coefficient at selected spatial frequency, solid line is cubic-spine-fitted curve, and gray dashed line is 1/7 criteria to determine resolutions. FIG. 12 panel D shows comparison of resolution analysis using different methods (n=10 datasets, including 4 datasets of AF647-labeled α-tubulin, 3 datasets of TOM20, and 3 datasets of Rtn4 in COS-7 cells), including lateral and axial estimated resolutions (EstXY of 11.0±1.4 nm and EstZ of 4.8±0.4 nm, red), FSC resolution (FSC of 19.8±2.0 nm, orange), imaging decorrelation resolution (Decor. of 14.2±1.3 nm, green), lateral and axial DFC resolutions (DFCXY of 20.8±3.1 nm and DFCZ of 16.7±2.5 nm, purple), and measured lateral and axial FWHMs of superposed single-molecule cluster (FWHMXY of 9.9+1.2 nm and FWHMZ of 5.1±0.3 nm, blue). Central line is mean, error bar is standard deviations, and gray dashed line is estimated isotropic 3D estimated resolution.

### Demonstration of 4Pi-BRAINSPOT in Mouse Brain Slices

FIG. 13 panel A shows reconstruction of AF647-labeled ChR2 in 50-μm thick mouse brain slices in FIG. 6 panel A, color-coded according to the single-molecule Z positions. FIG. 13 panels B-C show vertical cross-sections (top) of targeting dendritic spines (white dashed lines) in (Panel A). Normalized intensity profile (bottom) along white dashed lines. Yellow bar is localization histogram with 6-nm bins, red curve is fitted Gaussian functions to the histogram, gray dashed line is exclusion threshold, black solid line marks estimated feature FWHM, solid arrowhead indicates excluded isolated features, and dashed arrowhead indicates feature discontinuity. FIG. 13 panel D shows heatmap of image decorrelation resolutions by segmenting 2D projection of (A) into 7×7 subregions. FIG. 13 panel E shows comparison of resolution analysis using different methods, including lateral and axial estimated resolutions (EstXY of 14.4±3.7 nm and EstZ of 6.4±1.6 nm, n=3244 localizations, red), FSC resolution (FSC of 28.3±3.6 nm, n=81 subregions, orange), imaging decorrelation resolution (Decor. of 17.4±0.4 nm, n=81 subregions, green), lateral and axial DFC resolutions (DFCXY of 28.7 nm and DFCZ of 23.5 nm, purple), and measured lateral and axial FWHMs of superposed single-molecule cluster (FWHMXY of 12.8 nm and FWHMZ of 6.4 nm, cyan). Box is upper and lower quartiles, central line is median, error bar is furthest data point excluding outliers, gray dashed line is estimated isotropic 3D estimated resolution, and distribution is shown on the right side. FIG. 13 panels F-G show horizontal (Panel F) and vertical (Panel G) cross-sections (left) and normalized intensity profiles (right) of superposed single-molecule cluster. Red dot is single-molecule localization (n=3244 localizations), grayscale distribution is rendered cluster using 2D Gaussian blur with estimated FWHM, orange curve is fitted Gaussian function, and blue line marks estimated FWHM.

### Ambiguity in 4Pi-PSF Configurations

FIG. 14A panel A shows 4Pi-PSFs of channel p1 at z positions ranging from ±1 μm with vertical astigmatism variations of #1λ/2π (case 1, top row) and ∓1λ/2π (case 2, middle row) in the upper and lower pupil functions, respectively. Yellow and cyan boxes highlight identical PSFs with opposite Z positions in cases 1 and 2. Overlapping 4Pi-PSFs with axial symmetry ambiguity (bottom row) between case 1 (yellow) and case 2 with reversed sequence (cyan). FIG. 14A panel B shows 4Pi-PSFs of channel pl at z positions ranging from ±1 μm with vertical coma aberrations of −0.5 and 1.5λ/2π (case 3, top row) and −1.5 and 0.5λ/2π(case 4, middle row) in the upper and lower pupil functions, respectively. Cases 3 and 4 show identical PSFs at all Z positions. Overlapping 4Pi-PSFs with indistinguishable ambiguity (bottom row) between case 3 (yellow) and case 4 (cyan). FIG. 14B panel C shows Effect of covariant and contravariant objective misalignment in 4Pi-SMSN. FIG. 14B panel D shows 4Pi-PSFs of channel p1 at z positions ranging from ±1 μm with vertical coma aberrations of −0 and 4λ/2π (case 5, top row) and −2 and 2λ/2π (case 6, middle row) in the upper and lower pupil functions, respectively. Cyan dashed line highlights the translation between identical PSF patterns in cases 5 and 6. Overlapping 4Pi-PSFs with indistinguishable translated ambiguity (bottom row) between case 5 (yellow) and case 6 with translation (cyan).

### Demonstration of Spine Morphologies

FIG. 15 panels A-L show demonstrations of spine morphologies as horizontal cross-sections (left), vertical cross-sections of spine neck (middle), and vertical cross-sections of spine head (right), color-coded according to single-molecule axial positions (n=12 spines in FIG. 6 panel J). Yellow curve is the trace of algorithm from manual identification.

What is claimed is:

1. A system for achieving molecular resolution through tissues, the system comprising:
an interferometric apparatus; and
a control unit comprising one or more processors configured to:
receive interferometric tissue data from the interferometric apparatus, wherein the interferonic tissue data comprises one or more tissue-induced abberations;
apply a 4Pi in-situ point spread function (PSF) retrieval algorithm to the interferonic tissue data, thereby enabling direct retrieval of an aberrated, time-dependent in-situ model of single-molecule emission pattern from the interferonic tissue data, wherein the in-situ model reflects distortions, blurring, and/or partial coherence encountered during detection; and
minimize disparities between the interferonic tissue data and the in-situ model to thereby provide accurate molecular localizations in the presence of tissue-induced aberrations.

2. The system of claim 1, wherein the 4Pi in-situ PSF retrieval algorithm comprises segmenting multichannel 4Pi emission patterns to create a library of detected emission patterns.

3. The system of claim 2, wherein the segments, from stochastically blinking events at varying axial positions, serve as random samples of underlying in-situ interferometric (4Pi) PSF that are desired to be retrieved.

4. The system of claim 3, wherein to estimate axial positions of patterns in the library, reference PSFs are generated from coherent pupil functions, depicting interferometric amplitude and phase of wavefield superposition from the pupils of two opposing objectives.

5. The system of claim 4, wherein detected emission patterns are then assigned to specific axial groups according to their similarity to the reference PSFs.

6. The system of claim 5, wherein categorized patterns are then aligned and averaged within each group to generate experimental PSF observations.

7. The system of claim 6, wherein to retrieve individual pupil functions, a coherent 4Pi phase retrieval algorithm allows retrieval of the individual pupil functions for each objective from their coherent superpositions and updates the reference 4Pi-PSFs for the subsequent iteration.

8. The system of claim 7, wherein through sequential iterations, the model is refined to capture detailed coherent aberrations from both instrumental imperfections and biological specimens, representing in-situ interferometric emission patterns for accurate and precise single-molecule localization inside tissue specimens.

9. The system of claim 1, wherein the interferonic tissue data comprises data from brain tissue slices that are at least 50-μm thick.

10. The system of claim 9, wherein the system provides an isotropic 3D resolution of sub-15 nm in the brain tissue slices while minimizing distortions and artifacts.

11. A method for achieving molecular resolution through tissues, the method comprising:
receiving to a processor interferometric tissue data from an interferometric apparatus, wherein the interferonic tissue data comprises one or more tissue-induced abberations;
applying via the processor a 4Pi in-situ point spread function (PSF) retrieval algorithm to the interferonic tissue data, thereby enabling direct retrieval of an aberrated, time-dependent in-situ model of single-molecule emission pattern from the interferonic tissue data, wherein the in-situ model reflects distortions, blurring, and/or partial coherence encountered during detection; and
minimizing via the processor disparities between the interferonic tissue data and the in-situ model to thereby provide accurate molecular localizations in the presence of tissue-induced aberrations.

12. The method of claim 11, wherein the 4Pi in-situ PSF retrieval algorithm comprises segmenting multichannel 4Pi emission patterns to create a library of detected emission patterns.

13. The method of claim 12, wherein the segments, from stochastically blinking events at varying axial positions, serve as random samples of underlying in-situ interferometric (4Pi) PSF that are desired to be retrieved.

14. The method of claim 13, wherein to estimate axial positions of patterns in the library, reference PSFs are generated from coherent pupil functions, depicting interferometric amplitude and phase of wavefield superposition from the pupils of two opposing objectives.

15. The method of claim 14, wherein detected emission patterns are then assigned to specific axial groups according to their similarity to the reference PSFs.

16. The method of claim 15, wherein categorized patterns are then aligned and averaged within each group to generate experimental PSF observations.

17. The method of claim 16, wherein to retrieve individual pupil functions, a coherent 4Pi phase retrieval algorithm allows retrieval of the individual pupil functions for each objective from their coherent superpositions and updates the reference 4Pi-PSFs for the subsequent iteration.

18. The method of claim 17, wherein through sequential iterations, the model is refined to capture detailed coherent aberrations from both instrumental imperfections and biological specimens, representing in-situ interferometric emission patterns for accurate and precise single-molecule localization inside tissue specimens.

19. The method of claim 11, wherein the interferonic tissue data comprises data from brain tissue slices that are at least 50-μm thick.

20. The method of claim 19, wherein the method provides an isotropic 3D resolution of sub-15 nm in the brain tissue slices while minimizing distortions and artifacts.
